(19) United States
(12) Patent Application Publication
(10) Pub. No.: US 2024/0180849 A1
(43) Pub. Date: Jun. 6, 2024

(54) IMMUNOFUNCTIONAL CARRIER, METHODS OF USES, AND COMPOSITION MATTERS AS AN ANTITUMOR IMMUNOTHERAPY

(71) Applicant: Purdue Research Foundation, West Lafayette, IN (US)

(72) Inventors: Yoon Yeo, West Lafayette, IN (US); Jianping Wang, West Lafayette, IN (US); Fanfei Meng, West Lafayette, IN (US)

(73) Assignee: Purdue Research Foundation, West Lafayette, IN (US)

(21) Appl. No.: 18/285,439

(22) PCT Filed: Apr. 12, 2022
(86) PCT No.: PCT/US22/24332

§ 371 Date: Oct. 3, 2023

Related U.S. Application Data
(60) Provisional application No. 63/177,150, filed on Apr. 20, 2021.

(51) Int. Cl. A61K 9/70; A61K 45/06; A61K 47/59; A61K 49/00; A61P 35/00; C12N 15/113

(52) U.S. Cl. CPC A61K 9/70; A61K 45/06; A61K 47/59; A61K 49/0054; A61P 35/00; C12N 15/1138; A61K 2123/00; C12N 2310/141

(57) ABSTRACT

The present disclosure generally relates to a composition matter and a method for cancer treatment. In particular, a composition matter as an antitumor immunotherapy comprising a polyethyleneimine derivative as an immunoadjuvant, a chemotherapeutic drug, and optional components such as a microRNA, messenger RNA, plasmid DNA, siRNA, oligonucleotide, or a cyclic dinucleotide. A method for treating a subject with a cancer by administrating a therapeutic effective amount of a composition comprising a polyethyleneimine derivative and an antitumor agent to the subject in need of relief from said cancer is also within the scope of this disclosure.

### CROSS REFERENCE TO RELATED APPLICATIONS

This present patent application relates to and claims the priority benefit of U.S. Provisional Application Ser. No. 63/177,150, filed Apr. 20, 2021, the content of which is hereby incorporated into this disclosure by reference in its entirety.

### GOVERNMENT SUPPORT CLAUSE

This invention was made with government support under CA232419 and CA258737 awarded by the National Institutes of Health. The government has certain rights in the invention.

### STATEMENT OF SEQUENCE LISTING

A computer-readable form (CRF) of the Sequence Listing is submitted concurrently with this application. The file, entitled 69447-02_Seq_Listing_ST25_txt, having a size of 2 kb, is generated on Mar. 30, 2022. Applicant states that the content of the computer-readable form is the same and the information recorded in computer readable form is identical to the written sequence listing.

### TECHNICAL FIELD

The present disclosure generally relates to a composition matter and a method for cancer treatment. In particular, a composition matter as an antitumor immunotherapy comprising a polyethyleneimine derivative as an immunoadjuvant, a chemotherapeutic drug, and optional components of microRNA, siRNA, or an oligonucleotide, a nucleic acid, or a cyclic dinucleotide.

### BACKGROUND AND SUMMARY

This section introduces aspects that may help facilitate a better understanding of the disclosure. Accordingly, these statements are to be read in this light and are not to be understood as admissions about what is or is not prior art.

Since the success of immune checkpoint blockade (ICB)¹ and chimeric antigen receptor T cell therapy², over a dozen immunotherapies have been approved in the past few years, and thousands of immunotherapeutics are currently in the development pipeline³. However, immunotherapy benefits only a small fraction of cancer patients with identified tumor antigens and/or well-accessible tumors⁴. New therapeutic strategies are needed to improve the efficacy of immunotherapeutics in broader patient populations with hard-to-reach, unidentified tumors.

An approach gaining interest in the immuno-oncology community is to treat locatable and accessible tumors locally and stimulate antitumor immunity in situ to exert systemic effects against distant tumors^{5,6}. The rationale of local immunotherapy is that properly treated tumor cells serve as a depot of tumor antigens and induce systemic immune response against tumor via circulating immune cells⁶. By confining therapeutics in tumors, local immunotherapy can avoid systemic side effects, such as immune-related adverse events, which have limited the utility of traditional approaches⁷. Based on this premise, the number of local immunotherapy trials has grown exponentially in the past 10 years, with more than 40 early phase clinical trials in progress⁸.

For effective local immunotherapy, several events need to be coordinated coherently,

including in-situ generation of tumor-associated antigens (TAAs), activation of antigen-presenting cells (APCs), infiltration of immune cells to the tumor microenvironment (TME), and the maintenance of immunoactive TME. The complexity of antitumor immune responses requires combinations of agents with distinct mechanisms of action. For example, chemotherapeutic drugs are used to induce immunogenic cell death (ICD) to generate TAAs and release damage-associated molecular patterns (DAMPs)^{9,10}, which make the dying cells vulnerable to APC uptake⁹. Nucleic acids and nucleotides are frequently employed due to their diverse functions: small nucleotides can serve as potent immunoadjuvants¹¹, siRNA can be used to block immune checkpoints¹², and microRNA can regulate inflammatory cytokine production¹³.

For local delivery of multiple immunotherapeutics in cancer therapy, carrier selection is important in at least three aspects. First, a carrier can help retain immunotherapy locally to maximize pharmacological effects of therapeutic agents in tumors and prevent their systemic side effects¹⁴. Second, a carrier can ensure the colocalization of multiple agents¹⁵. For example, paclitaxel (PTX) inducing ICD⁹ and siRNA targeting immune checkpoint may be combined for complementary functions. A properly designed carrier can co-deliver the two drugs, which share little physicochemical features and would otherwise be difficult to colocalize. Third, a carrier engineered with an immunoadjuvant function can play an active role in triggering antitumor immunity^{16,17}, synergizing with immunostimulatory effects of therapeutic drugs. Nevertheless, it is not straightforward to develop an immunoactive local carrier of multiple drugs; earlier efforts to achieve this goal have relied on preformulation¹⁸ or prodrug formation of at least one of the components¹⁹, which needs to be tailored to individual drug.

Here, we develop a polyethyleneimine derivative (2E′), which activates immune cells and co-delivers hydrophobic immunogenic cell death inducers and immunomodulatory nucleic acids/nucleotides. A single local administration of 2E′ or its combination with paclitaxel and PD-L1 siRNA or cyclic dinucleotide induces strong antitumor immunity, resulting in immediate regression of large established tumors, tumor-free survival, and the resistance to rechallenge and metastasis in different models. This study supports that effective in-situ induction of antitumor immunity can lead to systemic protection from distant and recurrent diseases, where 2E′ plays multiple roles as a simple and versatile carrier of immunotherapeutic.

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures, and wherein:

FIGS. 1A-1N. Characterization and immunostimulatory activities of 2E′. FIG. 1A. Structure of LCA-PEI conjugate (2E′). FIG. 1B. TEM image of 2E′ assembled in water. Scale bar: 100 nm. FIG. 1C. In vitro cytotoxicity of 2E′ in BMDCs, CT26 and B16F10 cells. (n=3 or 6 replicates of a representative batch, mean±standard deviation, SD). FIG. 1D. Maturation marker expression on JAWSII DCs after incubation with PEI and 2E′ (7 μg/mL) for 24 h. (n=3 replicates of a representative batch, mean±SD). FIG. 1E. TNF-α and IL-1β secretion by BMDCs after incubation with 2E′ and PEI for 24 h (n=3 or 4 replicates of a representative batch, mean±SD). FIG. 1f. TLR-5 reporter cell activation by 2E′ and PEI indicated by the production of secreted embryonic alkaline phosphatase (SEAP) (n=3 or 4 replicates of a representative batch, mean±SD). FIG. 1g. 2E′ and PEI-induced CRT exposure on CT26 and B16F10 cells (n=3 replicates of a representative batch, mean±SD). FIG. 1h. Schedule of phagocytosis assay. FIG. 1i. Flow cytometric analysis of the phagocytosis of CT26 cells by BMDCs without (−) or with (+) 7 μg/mL 2E′. % CT26⁺ BMDCs: fraction of BMDCs taking up CT26 cells; % phagocytosed CT26: fraction of CT26 cells taken up by BMDCs (n=4 or 5 replicates of a representative batch, mean±SD). For calculation of the two fractions. FIG. 1j. Representative confocal images of CT26 cells incubated with BMDCs without (−) or with (+) 7 μg/mL 2E′ (Left) and the calculated Pearson's correlation coefficient, indicating colocalization of CT26 cells and BMDCs (Right). CT26 cells were stained with Celltracker Green (green), and BMDC cells with CellTracker DeepRed (red). Scale bars: 50 μm. Arrow heads indicate colocalization of CT26 cells and BMDCs. Five images were taken from two replicate wells. FIG. 1k. TEM images of 2E′/PTX with different weight ratios. Scale bars: 200 nm. FIG. 11. PTX release profile from 2E′/PTX in water solution containing 0.2% Tween-80 (n=3 replicates of a representative batch, mean±SD). FIG. 1m. Top: gel electrophoresis of 2E′/siPD-L1 complexes at various weight ratios of 2E′ to siPD-L1. All lanes contain complexes equivalent to 1 μg siRNA. Bottom: confocal image of CT26 cells after incubation with 2E′/Cy3-labelled siPDL-1 (1.5:1, w/w) for 4 h at a dose equivalent to 2.66 μg/mL siPD-L1 (blue: nuclei, green: lysosome, red: siPD-L1), scale bar: 10 μm. FIG. 1n. Top: schedule of PD-L1 silencing assay. Bottom: western blot of PD-L1 expression in IFN-γ-activated B16F10 and CT26 cells after treatment with 2E′/siNeg or 2E′/siPD-L1 (4 μg/mL 2E′ and 2.66 μg/mL siPD-L1) (n=3 independent tests, mean±SD). p-values were calculated by Tukey's multiple comparisons test following ordinary one-way ANOVA (d), Šídák's multiple comparisons test following two-way ANOVA (e, f, g) or one-way ANOVA (n), and two-tailed unpaired t-test (i, j, n).

FIGS. 2A-2G. Characterization of 2E′/PTX/siPD-L1. FIG. 2a. Gel electrophoresis of 2E′/PTX/siPD-L1 complexes at various weight ratios of 2E′/PTX to siPD-L1. All lanes contain complexes equivalent to 1 μg siPD-L1. FIG. 2b. TEM image of 2E′/PTX/siPD-L1. Scale bar: 200 nm. FIG. 2c. Cytotoxicity of 2E′, PTX, 2E′/PTX and 2E′/PTX/siPD-L1 to CT26 cells, BMDCs, and splenocytes. n=4-6 replicates of a representative batch, mean±SD. p-values were calculated by Šídák's multiple comparisons test following two-way ANOVA. Antitumor effects of 2E′/PTX/siPD-L1 on CT26 tumors. FIG. 2d. Schedule of CT26 tumor inoculation in Balb/c mice, treatment injection, and rechallenges. FIG. 2e. Individual growth curves of tumors treated with 2E′, 2E′/PTX, 2E′/PTX/siNeg or 2E′/PTX/siPD-L1 consisting of 1 mg 2E′, 0.2 mg/mL PTX, and 0.67 mg siRNA by intratumoral (IT) injection (n=8 per group). FIG. 2f. Individual growth curves of the 1^{st} rechallenged tumors in tumor-free mice after a single intratumoral injection. FIG. 2g. Individual growth curves of the 2^{nd} rechallenged tumors in tumor-free mice resisting the 1^{st} tumor challenge. CR: complete regression.

FIGS. 3A-3F. Effects of a single intratumoral injection of 2E′/PTX/siPD-L1 on growth and immunophenotype of B16F10 tumors. FIG. 3a. Schedule of B16F10 tumor inoculation in C57BL/6 mice and treatment injection. FIG. 3b. Individual growth curves of tumors treated with D5W (n=4), 2E′ (n=5), 2E′/PTX (n=5), 2E′/PTX/siNeg (n=4) or 2E′/PTX/siPD-L1 (n=5) consisting of 1 mg 2E′, 0.2 mg PTX and 0.67 mg siRNA by intratumoral (IT) injection and body weight change after the treatment. FIG. 3c. Immune cell (CD11c⁺DCs, CD11c⁺CD40⁺, CD11c⁺CD86⁺ mature DCs, F4/80⁺ macrophages, CD80⁺ M1-like macrophages, CD206⁺ M2-like macrophages, Ly6C⁺ monocytic and Ly6G⁺ neutrophilic MDSCs and T cells) population in TME on day 7 post-treatment. FIG. 3d. PD-L1 expression on tumor cells (CD45⁻ cells), lymphocytes (CD45⁺ cells), macrophages (CD45⁺F4/80⁺) and MDSCs (CD11b⁺Ly6C⁺ or CD11b⁺Ly6G⁺) in TME. FIG. 3e. IFN-γ secretion from splenocytes of B16F10@C57BL/6 mice treated with D5W (n=3), 2E′/PTX/siNeg (n=4) or 2E′/PTX/siNeg (n=4) without (−) or with (+) the stimulation of 5 μg/mL melanoma-specific antigen Trp2 peptide (SVYDFFVWL, SEQ ID NO:5). Each pair indicates individual mouse. FIG. 3f. The frequency of Trp2 (SVYDFFVWL, SEQ ID NO:5)-specific CD8⁺ T cells in the splenic CD3⁺ cells. Spleens were collected on day 7 after treatment (e, f). p-values were calculated by Dunnett's multiple comparisons test following one-way ANOVA (c,d), Šídák's multiple comparisons test following two-way ANOVA (e) and Tukey's multiple comparisons test following one-way ANOVA (f). Data: mean±SD.

FIGS. 4A-4C. Effects of a single intratumoral injection of 2E′/PTX/siPD-L1 on systemic anti-tumor effect (a) and immune memory in B16F10@CT57BL/6 tumor model (b/c). FIG. 4a. Schedule of bilateral B16F10 tumor inoculation in C57BL/6 mice and treatment injection; tumor growth curves and tumor sizes on day 11 post-treatment of treated and untreated tumors after a single treatment of D5W (n=6), 2E′/siPD-L1+PTX NC (n=8), 2E′/PTX/siNeg (n=9) or 2E′/PTX/siPD-L1 (n=9); mean±SD. p-values were calculated by Dunnett's multiple comparisons test following one-way ANOVA. Note that some of the 2E′/PTX/siPD-L1-treated mice were euthanized due to tumor ulceration despite the small tumor size. FIG. 4b. Schedule of B16F10 tumor inoculation in C57BL/6 mice, treatment injection, and rechallenge; individual growth curves of tumors treated with D5W (n=4) and 2E′/PTX/siPD-L1 (n=11); survival curves (p-value: vs. D5W by Log-rank (Mantel-Cox) test); and body weight change after treatment. CR: complete regression. FIG. 4c. Individual growth curves of tumors in age-matched naïve mice; survival curves of naïve and tumor-rechallenged mice (p-value: vs. naïve by Log-rank (Mantel-Cox) test). The treatments contained 1 mg 2E′, 0.2 mg PTX and 0.67 mg siPD-L1 (or siNeg).

FIGS. 5A-5C. Effects of a single administration of 2E′/PTX/siPD-L1 on the growth and metastasis of 4T1 tumors. FIG. 5a. Schedule of 4T1 tumor inoculation in Balb/c mice, treatment injection, and rechallenge; bioluminescence imaging of the 4T1 tumor-bearing Balb/c mice following the treatment with D5W (n=5), surgical resection (n=5), 2E′/PTX/siNeg (n=9), or 2E′/PTX/siPD-L1 (n=8). The complexes consisted of 1 mg 2E′, 0.2 mg PTX and 0.67 mg siRNA. Red arrow heads indicate lung metastasis. Signals in yellow circles are reflections of the strong signal of the neighboring mouse. FIG. 5b. Individual growth curves of 4T1 tumors in response to different treatments; the size of tumors on day 19 post-treatment (mean±SD; p-values by Dunn's multiple comparisons test following Kruskal-Wallis ANOVA); survival curves (p-values: vs. D5W by Log-rank (Mantel-Cox) test); and body weight change after treatment. CR: complete regression. FIG. 5c. Bioluminescence imaging of age-matched naïve mice and 2E′/PTX/siPD-L1-treated tumor-free mice after a rechallenge of live 4T1 cells; percentage of tumor-free mice following (re)challenge (p-value: vs. naïve by Log-rank (Mantel-Cox) test); representative images of lungs on day 47 after rechallenge with live 4T1 cells.

FIGS. 6A-6B. Effects of a single intratumoral injection of 2E′/PTX/CDN on growth of CT26 tumors and development of antitumor immunity. FIG. 6a. Schedule of CT26 tumor inoculation in Balb/c mice, treatment injection, and rechallenge; individual growth curves of tumors treated with D5W (n=5), PTX NC+CDN (n=8), 2E′/PTX (n=7) and 2E′/PTX/CDN (n=7); survival curves (p-values: vs. D5W by Log-rank (Mantel-Cox) test); and body weight change after treatment. The complexes consisted of 1 mg 2E′, 0.2 mg PTX and 20 μg CDN. CR: complete regression. FIG. 6b. Left: Individual growth curves of CT26 tumors in age-matched naïve mice; percentage of tumor-free mice following (re)challenge with CT26 cells (p-values: vs. naive by Log-rank (Mantel-Cox) test). Right: Growth curves of 4T1 (unrelated) tumors inoculated in surviving tumor-free mice (age-matched naïve mice: n=5, 2E′/PTX treated: n=3, 2E′/PTX/CDN treated: n=6), mean±SD, n.s .: not significant; percentage of tumor-free mice following challenge with 4T1 cells.

FIGS. 7A-7C show a single intratumoral injection of 2E′ or 2E′/PTX induces quick regression of tumor and antitumor immune responses in CT26@Balb/c model with bilateral tumors. FIG. 7a. Schedule of CT26 tumor inoculation in Balb/c mice and treatment injection. FIG. 7b. Individual growth curves of tumors treated with D5W, 2E′ (0.5 mg), 2E′ (1 mg) or 2E′/PTX (1 mg:0.2 mg) and the size of treated tumors on day 10 post-treatment (n=5 per group). CR: complete regression. FIG. 7c. Individual growth curves of untreated distant tumors and the size of distant tumors on day 10 post-treatment of primary tumors (n=5 per group). Mean±SD; p-values were calculated by Dunn's multiple comparisons test following Kruskal-Wallis one-way ANOVA.

FIGS. 8A-8C show a-single intratumoral injection of 2E′ or 2E′/PTX induces quick regression of tumor and antitumor immune responses in CT26@Balb/c model with a delayed 2^{nd} tumor challenge. FIG. 8a. Schedule of CT26 tumor inoculation in Balb/c mice and treatment injection. FIG. 8b. Individual growth curves of tumors treated with D5W (n=4), 2E′ (2.7 mg) (n=7) or 2E′/PTX (2.7 mg:0.4 mg) (n=7) and the size of treated tumors on day 22 post-treatment. Mean±SD; p-value was calculated by Dunn's multiple comparisons test following Kruskal-Wallis one-way ANOVA. FIG. 8c. Individual growth curves of untreated distant tumors. CR: complete regression.

FIGS. 9A-9C show antitumor effects of 2E′/PTX on CT26 tumors. FIG. 9a. Schedule of CT26 tumor inoculation in Balb/c mice, treatment injection, and rechallenge. FIG. 9b. Individual growth curves of tumors after a single IT injection of D5W, 2E′ (1.4 mg), or 2E′/PTX (1.4 mg:0.2 mg) and survival curve of treated mice (n=5 per group). p-values: vs. D5W by Log-rank (Mantel-Cox) test. FIG. 9c. Individual growth curves of rechallenged tumors in tumor-free mice after single treatment and percentage of tumor-free mice after re-challenge. CR: complete regression.

FIG. 10A shows a single intratumoral injection of 2E′/PTX reduces the recurrence of tumors and lung metastasis after incomplete surgical removal of primary tumors in orthotopic 4T1@Balb/c model. Survival curves of treated mice. FIGS. 10B and 10C show a single intratumoral injection of 2E′/PTX/siPD-L1 induces tumor regression and immunophenotype change in TDLNs of Balb/c mice with CT26 tumors. FIG. 10B. Individual growth curves of tumors treated with D5W (n=4), 2E′ (n=4), 2E′/PTX (n=4), 2E′/PTX/siNeg (n=4) or 2E′/PTX/siPD-L1 (n=5) consisting of 1 mg 2E′, 0.2 mg/mL PTX and 0.67 mg siRNA. FIG. 10C. T cell and DC cell populations in TDLNs on day 7 post-treatment. Mean±SD; p-values were calculated by Dunnett's multiple comparisons test following one-way ANOVA.

FIG. 11 shows 2E′ as a versatile carrier of hydrophobic drugs. 2E′ forms spherical particles upon assembly with various hydrophobic compounds, such as ICD inducers [carfilzomib (CFZ, selective proteasome inhibitor) and camptothecin (CPT, DNA topoisomerase inhibitor)]; hydrophobic fluorescence dyes: DiR (DiIC18(7); 1,1′-dioctadecyl-3.3.3′,3′-tetramethylindotricarbocyanine iodide); niflumic acid (a drug used for joint and muscular pain); probucol (anti-hyperlipidemic drug).

FIGS. 12A-12C show 2E′ as a carrier of nucleic acids. FIG. 12A. Gel electrophoresis of 2E′/PTX/pDNA complexes at various weight ratios of 2E′/PTX to pDNA. FIG. 12B. TEM images of 2E′/PTX/pDNA (1:0.4:0.7). Scale bars: 200 nm. FIG. 12C. Gel electrophoresis of 2E′/mRNA complexes at various weight ratios of 2E′ to mRNA. All lanes contain complexes equivalent to 1 μg pDNA or mRNA.

FIGS. 13A-13C show antitumor effects of 2E′/PTX/siPD-L1 in B16F10@C57BL/6 mice with bilateral tumors. Individual growth curves of treated (FIG. 13A) and untreated tumors (FIG. 13B). FIG. 13C: Survival curves and body weight change (mean±SD) after a single treatment of the following: D5W (n=6); 2E′/siPD-L1+PTX NC (n=8); 2E′/PTX/siNeg (n=9); or 2E′/PTX/siPD-L1 (n=9). Note that some of the 2E′/PTX/siPD-L1-treated mice were euthanized due to tumor ulceration despite the small tumor size.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%. 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls. The term “pharmaceutically acceptable carrier” is art-recognized and refers to a

pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μ/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” or “subject” includes human and non-human animals such as companion animals (dogs and cats and the like) and livestock animals. Livestock animals are animals raised for food production. The patient to be treated is preferably a mammal, in particular a human being.

As disclosed herein, a small interfering RNA (siRNA) is a unique term referring to double-stranded RNA molecules with 20-25 base pairs, involved in RNA interference. Definitions to those other RNAs can be found at Zhang. P. et al., J. Integr. Bioinform. 2019 Sep; 16(3): 20190027.

In some illustrative embodiments, this present disclosure relates to a composition matter as an antitumor immunotherapy or a diagnosis tool comprising a polyethyleneimine derivative as an immunoadjuvant and a chemotherapeutic drug or a hydrophobic molecule.

In some illustrative embodiments, this present disclosure relates to a composition matter as an antitumor immunotherapy or a diagnosis tool as disclosed herein, wherein said composition further comprising a microRNA, messenger RNA, plasmid DNA, small interfering RNA (siRNA), oligonucleotide, or a cyclic dinucleotide.

In some illustrative embodiments, this present disclosure relates to a composition matter as an antitumor immunotherapy or a diagnosis tool as disclosed herein, wherein said siRNA is PD-L1 siRNA.

In some illustrative embodiments, this present disclosure relates to a composition matter as an antitumor immunotherapy or a diagnosis tool as disclosed herein, wherein said chemotherapeutic drug or a hydrophobic molecule is paclitaxel, sorafenib, itraconazole, docetaxel, doxorubicin, bortezomib, carfilzomib, camptothecin, cisplatin, oxaliplatin, cytarabine, vincristine, irinotecan, amphotericin, niflumic acid, probucol, indomethacin, gemcitabine, or a pharmaceutically acceptable salt thereof, or a hydrophobic dye or a salt thereof.

In some illustrative embodiments, this present disclosure relates to a composition matter as an antitumor immunotherapy or a diagnosis tool as disclosed herein, wherein said hydrophobic dye is a hydrophobic fluorescent dye comprising DiR′; DiIC18(7) (1,1′-Dioctadecyl-3,3,3′,3′ Tetramethylindotricarbocyanine Iodide), Cyanine7, Cyanine 5, or an acceptable salt thereof.

In some illustrative embodiments, this present disclosure relates to a composition matter as an antitumor immunotherapy or a diagnosis tool as disclosed herein, wherein said polyethyleneimine derivative is a modified/conjugated polyethyleneimine by lithocholic acid (LCA), cholic acid, glycocholic acid, taurocholic acid, deoxycholic acid, chenodeoxycholic acid, glycochenodeoxycholic acid, taurochenodeoxycholic acid, or an acceptable salt thereof.

In some illustrative embodiments, this present disclosure relates to a composition matter as an antitumor immunotherapy or a diagnosis tool as disclosed herein, wherein said polyethyleneimine has a molecular weight range of about 2,500 Da to 250,000 Da.

In some illustrative embodiments, this present disclosure relates to a composition matter as an antitumor immunotherapy, or a diagnosis tool as disclosed herein, wherein said composition matter is administered intratumorally.

In some illustrative embodiments, this present disclosure relates to a composition matter as an antitumor immunotherapy, or a diagnosis tool as disclosed herein, wherein said composition matter is administered systemically.

In some illustrative embodiments, this present disclosure relates to a pharmaceutical composition comprising the composition matter as disclosed herein, together with one or more diluents, excipients, or carriers.

In some other illustrative embodiments, this present disclosure relates to a method for treating a subject with cancer comprising the step of administrating a therapeutic effective amount of a composition comprising a polyethyleneimine derivative as an immunoadjuvant and an antitumor agent to the subject in need of relief from said cancer.

In some other illustrative embodiments, this present disclosure relates to a method for treating a subject with cancer comprising the step of administrating a therapeutic effective amount of a composition comprising a polyethyleneimine derivative as an immunoadjuvant and an antitumor agent to the subject in need of relief from said cancer as disclosed herein, wherein said method further comprising a microRNA, messenger RNA, plasmid DNA, small interfering RNA (siRNA), oligonucleotide, or a cyclic dinucleotide.

In some other illustrative embodiments, this present disclosure relates to a method for treating a subject with cancer comprising the step of administrating a therapeutic effective amount of a composition comprising a polyethyleneimine derivative as an immunoadjuvant and an antitumor agent to the subject in need of relief from said cancer as disclosed herein, wherein said siRNA is PD-L1 siRNA.

In some other illustrative embodiments, this present disclosure relates to a method for treating a subject with cancer comprising the step of administrating a therapeutic effective amount of a composition comprising a polyethyleneimine derivative as an immunoadjuvant and an antitumor agent to the subject in need of relief from said cancer as disclosed herein, wherein said chemotherapeutic drug is a hydrophobic chemotherapeutic molecule.

In some other illustrative embodiments, this present disclosure relates to a method for treating a subject with cancer comprising the step of administrating a therapeutic effective amount of a composition comprising a polyethyleneimine derivative as an immunoadjuvant and an antitumor agent to the subject in need of relief from said cancer as disclosed herein, wherein said hydrophobic chemotherapeutic drug comprises paclitaxel, sorafenib, itraconazole, docetaxel, doxorubicin, bortezomib, carfilzomib, camptothecin, cisplatin, oxaliplatin, cytarabine, vincristine, irinotecan, amphotericin, niflumic acid, probucol, indomethacin, gemcitabine, or a pharmaceutically acceptable salt thereof.

In some other illustrative embodiments, this present disclosure relates to a method for treating a subject with cancer comprising the step of administrating a therapeutic effective amount of a composition comprising a polyethyleneimine derivative as an immunoadjuvant and an antitumor agent to the subject in need of relief from said cancer as disclosed herein, wherein said polyethyleneimine derivative is a wherein said polyethyleneimine derivative is a modified/conjugated polyethyleneimine by lithocholic acid (LCA), cholic acid, glycocholic acid, taurocholic acid, deoxycholic acid, chenodeoxycholic acid, glycochenodeoxycholic acid, taurochenodeoxycholic acid, or an acceptable salt thereof.

In some other illustrative embodiments, this present disclosure relates to a method for treating a subject with cancer comprising the step of administrating a therapeutic effective amount of a composition comprising a polyethyleneimine derivative as an immunoadjuvant and an antitumor agent to the subject in need of relief from said cancer as disclosed herein, wherein said polyethyleneimine has a molecular weight range of about 2,500 Da to about 250,000 Da.

In some other illustrative embodiments, this present disclosure relates to a method for treating a subject with cancer comprising the step of administrating a therapeutic effective amount of a composition comprising a polyethyleneimine derivative as an immunoadjuvant and an antitumor agent to the subject in need of relief from said cancer as disclosed herein, wherein said composition matter is administered intratumorally or systemically.

In some other illustrative embodiments, this present disclosure relates to a method for treating a subject with cancer comprising the step of administrating a therapeutic effective amount of a composition comprising a polyethyleneimine derivative as an immunoadjuvant and an antitumor agent to the subject in need of relief from said cancer as disclosed herein, wherein said composition may assume in a filament form.

In some other illustrative embodiments, this present disclosure relates to a composition matter for diagnosis purpose comprising a polyethyleneimine derivative and a hydrophobic dye.

In some other illustrative embodiments, this present disclosure relates to a composition matter for diagnosis purpose comprising a polyethyleneimine derivative and a hydrophobic dye, wherein said hydrophobic dye comprises DiR′; DiIC18(7) (1,1′-Dioctadecyl-3,3,3′,3′ Tetramethylindotricarbocyanine Iodide), Cyanine7, Cyanine 5, or an acceptable salt thereof.

For effective local immunotherapy, several events need to be coordinated coherently, including in-situ generation of tumor-associated antigens (TAAs), activation of antigen-presenting cells (APCs), infiltration of immune cells to the tumor microenvironment (TME), and the maintenance of immunoactive TME. The complexity of antitumor immune responses requires combinations of agents with distinct mechanisms of action. For example, chemotherapeutic drugs are used to induce immunogenic cell death (ICD) to generate TAAs and release damage-associated molecular patterns (DAMPs)^{9,10}, which make the dying cells vulnerable to APC uptake⁹. Nucleic acids and nucleotides are frequently employed due to their diverse functions: small nucleotides can serve as potent immunoadjuvants¹¹, siRNA can be used to block immune checkpoints¹², and microRNA can regulate inflammatory cytokine production¹³.

For local delivery of multiple immunotherapeutics in cancer therapy, carrier selection is important in at least three aspects. First, a carrier can help retain immunotherapy locally to maximize pharmacological effects of therapeutic agents in tumors and prevent their systemic side effects¹⁴. Second, a carrier can ensure the colocalization of multiple agents¹⁵. For example, paclitaxel (PTX) inducing ICD⁹ and siRNA targeting immune checkpoint may be combined for complementary functions. A properly designed carrier can co-deliver the two drugs, which share little physicochemical features and would otherwise be difficult to colocalize. Third, a carrier engineered with an immunoadjuvant function can play an active role in triggering antitumor immunity^{16,17}, synergizing with immunostimulatory effects of therapeutic drugs. Nevertheless, it is not straightforward to develop an immunoactive local carrier of multiple drugs; earlier efforts to achieve this goal have relied on preformulation¹⁸ or prodrug formation of at least one of the components¹⁹, which needs to be tailored to individual drug.

Here, we have developed a new carrier of immunotherapeutics for local application, based on an amphiphilic modification of polyethyleneimine (PEI), a polymeric gene carrier²⁰ and a toll-like receptor (TLR)-5 agonist²¹. We conjugated lithocholic acid (LCA), a hydrophobic bile acid with an immunostimulatory effect on APCs²², to the flank of PEI to accommodate hydrophobic drugs and enhance the immunoadjuvant function. We demonstrate that the PEI-LCA conjugate (2E′) forms supramolecular assemblies in water, loads both hydrophobic drugs and nucleic acids by simple mixing, and stimulates APCs to complement the activities of the active ingredients. A single intratumoral administration of 2E′, along with PTX, induces immediate regression of tumors and generates antitumor immunity in the CT26 tumor model. Additional incorporation of siRNA targeting PD-L1 (siPD-L1) or cyclic dinucleotide (CDN) further enhances the immunostimulatory effects. leading to the regression of large established tumors and tumor-free survival in multiple models after a single administration. The local induction of antitumor immunity activates systemic antitumor immunity and immune memory to protect surviving animals from tumor rechallenge and metastasis. The potent antitumor activity of this immunoactive complex demonstrates the importance of a rationally-designed drug carrier and supports the feasibility of treating hard-to-reach tumors by effective local immunotherapy.

### Results

### 2E′ (Polyethyleneimine-Lithocholic Acid Conjugate) is Less Toxic to Dendritic Cells (DCs), Selectively Toxic to Cancer Cells, and Safer In Vivo Than PEI.

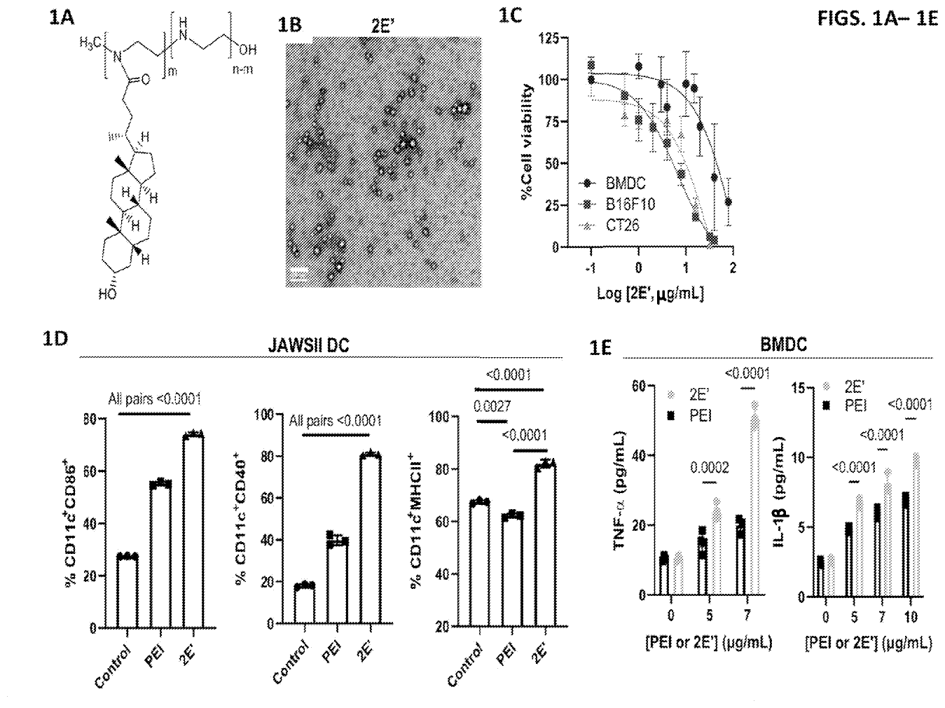

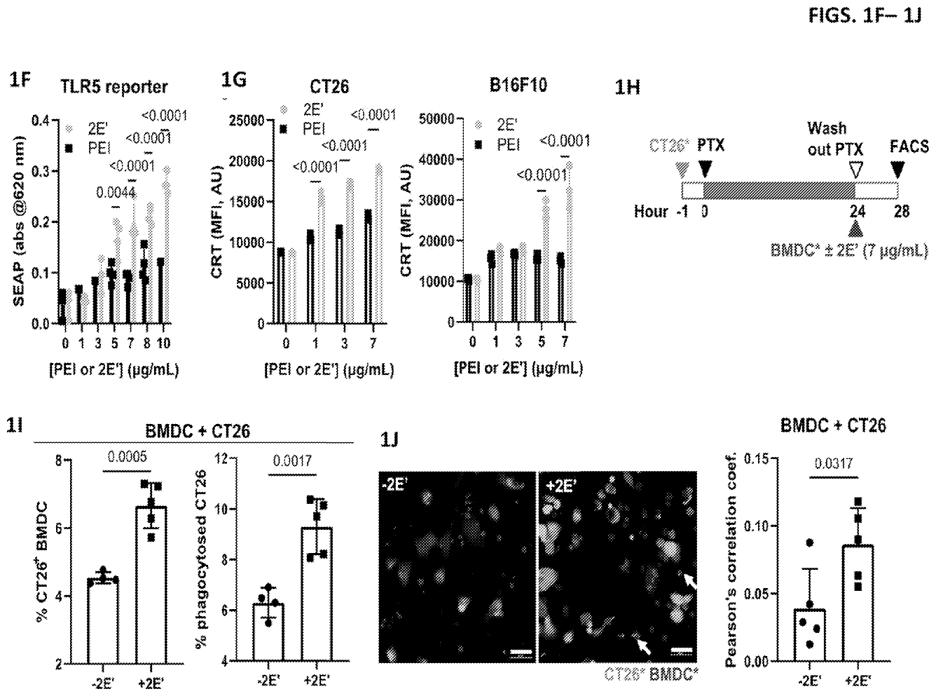

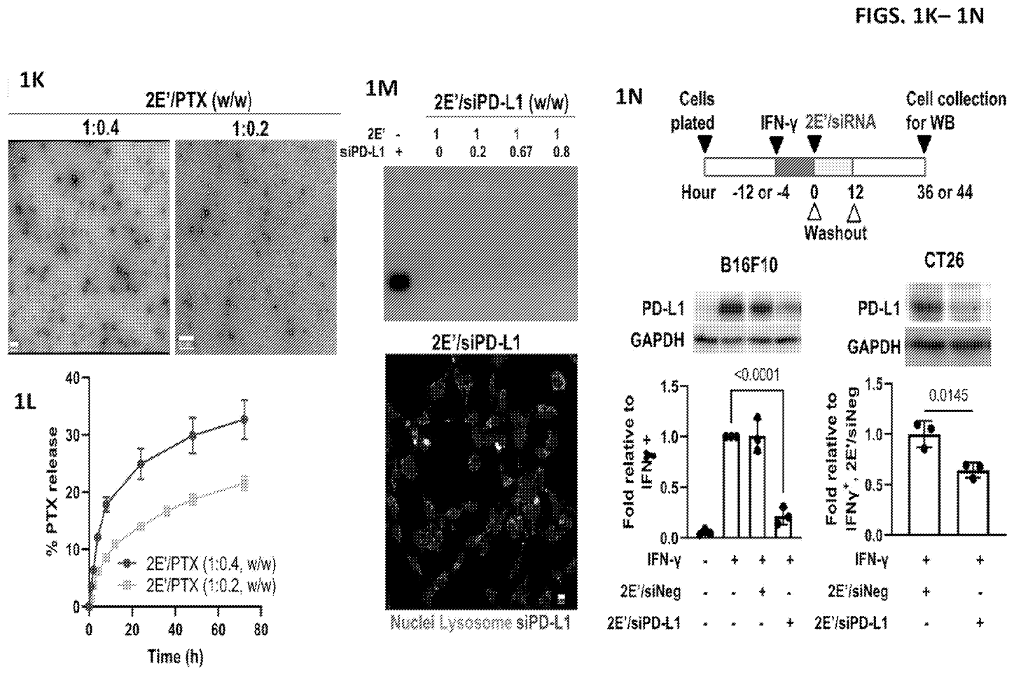

2E′ (FIG. 1a) was synthesized by conjugating LCA to PEI via carbonyldiimidazole in 2:1 molar ratio²³. In aqueous medium, 2E′ formed a nanoparticle assembly (FIG. 1b) due to the LCA moieties, with a critical assembly concentration of 2.6 μg/mL. 2E′ was more toxic to CT26 and B16F10 cells than to bone marrow-derived DCs (BMDCs) (FIG. 1c). Since cancer cells tend to display relatively more anionic surface than normal cells^{24,25}, the selective toxicity of 2E′ may be explained by the affinity for cancer cells based on the charge difference. The literature supports that selective toxicity to cancer cells is a shared feature of cationic polymers (26, 27); indeed, both 2E′ and PEI showed greater toxicity to CT26 cells than normal splenocytes. Despite the similarity in cytotoxicity profiles, 2E′ and PEI showed a notable difference upon intratumoral injection. All mice receiving PEI at 2.74 mg/mouse (7/7) died in 48 h, whereas none (0/7) died in the 2E′-treated group (2E′ at 2.74 mg/mouse). PEI is known to cause a shock associated with lung damage when administered systemically²⁶. To test if the differential in vivo toxicity resulted from the extent of their systemic absorption, we administered Cy7-labeled PEI and 2E′ intratumorally to CT26 tumor and monitored their retention by whole-body fluorescence imaging. 2E′ disappeared from the injection site more slowly than PEI, likely due to the nanoparticle configuration (FIG. 1b). The relatively slow absorption may have attenuated in vivo toxicity of intratumorally-injected 2E′.

2E′ Serves as an Immunoadjuvant and Enhances Cancer Cell Uptake by APCs, Carries PTX and siPD-L1, and Induces Antitumor Response in CT26@Balb/c and 4T1@Balb/c Models.

PEI is a known agonist of TLR-5²¹ and Nlrp3 inflammasome^{17,27}. To test if 2E′ retained the immunostimulatory effect of PEI, we applied 2E′ to bone marrow-derived myeloid cells and JAWSII DCs. 2E′ induced the maturation of BMDCs and JAWSII DCs (FIG. 1d) and promoted the production of tumor necrosis factor-α (TNF-α) and interleukin-1β (IL-1β), an indicator of inflammasome activation²⁸, from BMDCs and BMDMs (FIG. 1c). A colorimetric assay with a TLR reporter cell line showed that 2E′ activated TLR-5 signaling (FIG. 1f). Neither 2E′ nor PEI activated TLR-4 signaling at comparable concentrations, excluding potential artifact of endotoxin contamination. Additionally, CT26 and B16F10 cells treated with 2E′ exposed the cell surface calreticulin (CRT) (FIG. 1g), a sign of ICD²⁹. In all the tests, 2E′ induced greater responses than PEI, which may be attributable to the immunostimulatory activity of LCA pendants²². We have confirmed that LCA stimulates BMDC maturation in a dose dependent manner and shown that LCA increases the immunostimulatory effect of PEI when combined with PEI. Nevertheless, the additive effect of LCA and PEI does not fully explain the improved immunostimulatory effects of 2E′, since the difference between PEI and 2E′ was greater than the difference of PEI and LCA+PEI. The superior activity of 2E′ may also be ascribed to the formation of NP configuration, which promotes the solubilization and cellular uptake of water-insoluble LCA. Based on the immunostimulatory activity of 2E′, we tested if 2E′ enhanced the ability of APCs to take up tumor antigens (e.g., tumor cells undergoing ICD^{30,31}). We co-cultured DCs or BMDMs with CT26 or B16F10 cells, which had been pre-treated with PTX, a type-I ICD inducer^{9,30,32}, with or without 2E′ (FIG. 1h), and quantified the APC uptake of cancer cells by flow cytometry. The fractions of DCs or BMDMs taking up cancer cells and the cancer cells taken up by DCs or BMDMs substantially increased with 2E′ (FIG. 1i, 1j). These results suggest that 2E′ facilitate the initial step of antitumor immune response by serving as an immunoadjuvant and enhancing the APC uptake of tumor antigens.

Due to the hydrophobicity of the LCA flank and positive charge of PEI backbone, 2E′ encapsulated PTX and complexed with siPD-L1. PTX-encapsulated 2E′ (2E′/PTX) particles were spherical and measured to be ˜20 nm in diameter (FIG. 1k), similar to 2E′. At a weight ratio of 1:0.4 (2E′:PTX), 2E′/PTX had an average PTX loading of 23.4±6.3 wt% (81.9% of the total used PTX) and released 32% of the encapsulated PTX in aqueous solution over 72 h (FIG. 11). 2E′/PTX (1:0.2, w/w) with a greater 2E′ content released PTX more slowly (21% in 72 h), suggesting that the hydrophobic LCA clusters controlled PTX release. One part of 2E′ complexed with as much as 0.67 part of siPD-L1, forming a complete complex that excluded ethidium bromide (FIG. 1m). 2E′/siPD-L1 entered CT26 cells as efficiently as Lipofectamine2000/siPD-L1 complex (L2k/siPD-L1) and inhibited PD-L1 expression on interferon-γ (IFN-γ)-stimulated B16F10 and CT26 cells (FIG. 1n).

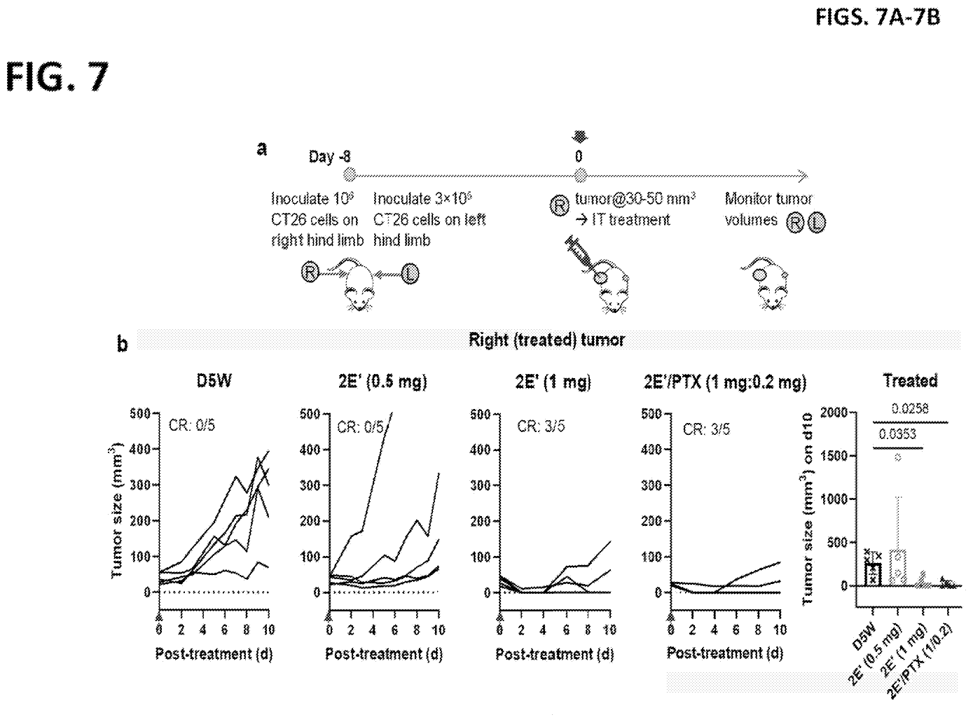

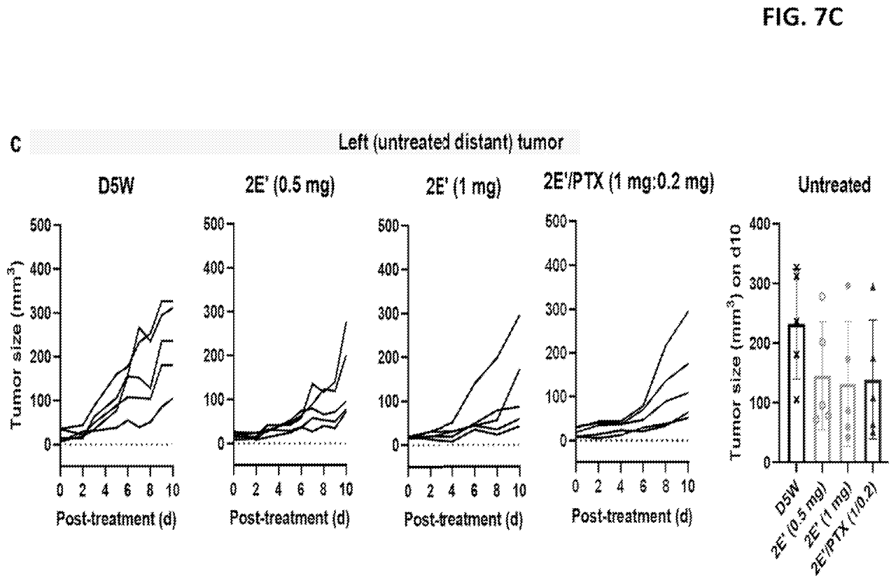

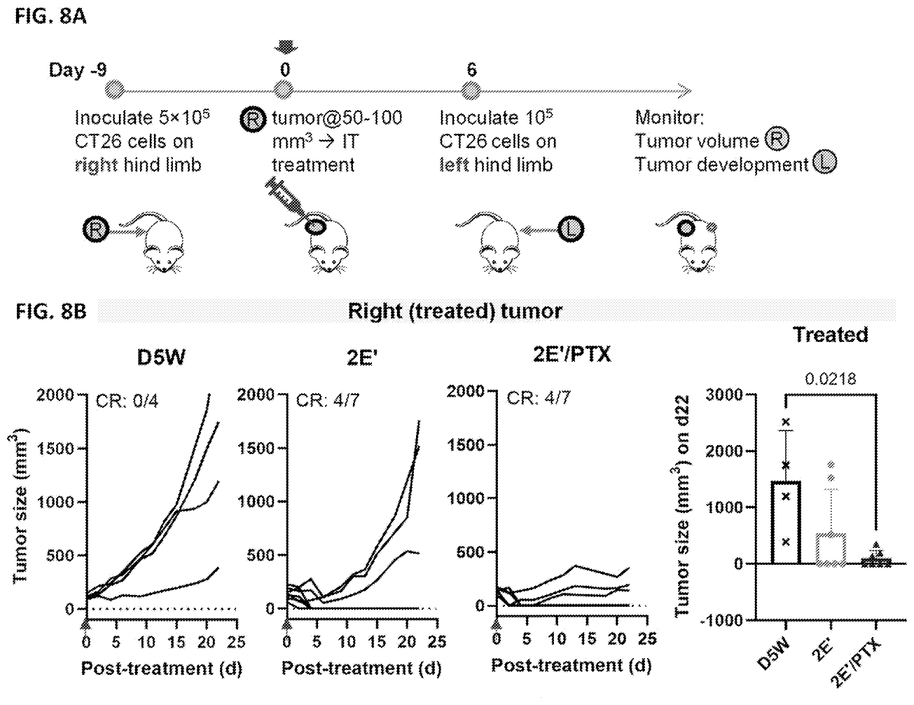

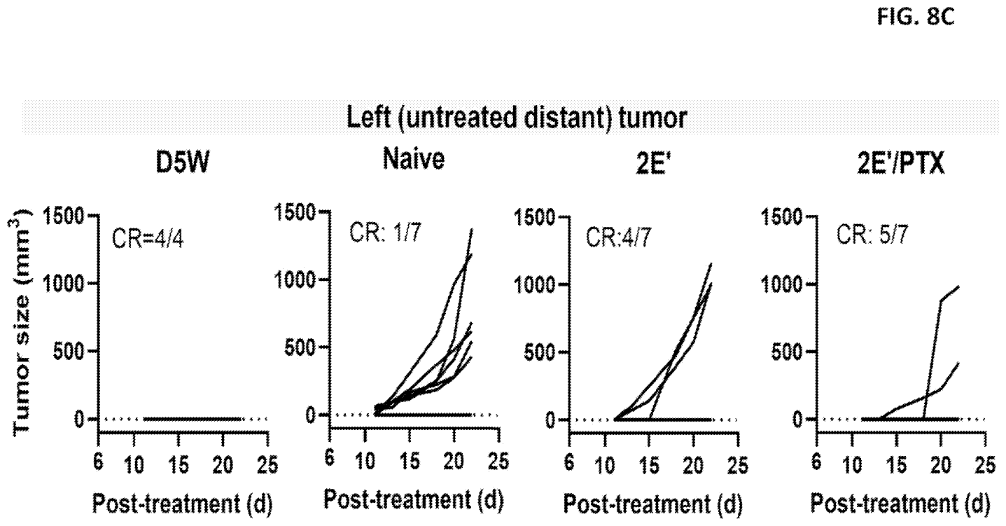

We performed a series of in-vivo studies to test if locally administered 2E′ or 2E′/PTX had an antitumor effect and induced systemic antitumor immunity. First, we administered 2E′ or 2E′/PTX to one of the bilateral CT26 tumors in Balb/c mice and observed the effects on treated and untreated distant tumors (abscopal effect) (FIG. 7a). A single intratumoral injection of 2E′ (1 mg per mouse) or 2E′/PTX (1 mg 2E′+0.2 mg PTX per mouse) induced significant attenuation or eradication of the treated tumor, with 60% of the mice in each group showing complete regression (CR) of the treated tumors (FIG. 7b). For untreated distant tumors, 2E′ or 2E′/PTX groups showed apparently slower tumor growth than the 5% dextrose (D5W) vehicle group, but the difference was insignificant (FIG. 7c). Second, we tested 2E′ and 2E′/PTX at a higher dose (2.7 mg 2E′ or 2.7 mg 2E′+0.4 mg PTX per mouse) with longer monitoring (FIG. 8a). This time, we inoculated the distant tumor 6 days after the treatment of primary tumor to avoid excessive growth before developing antitumor immunity. Both 2E′ and 2E′/PTX inhibited the growth of primary tumors in 57% of the mice, while no mouse in the D5W-treated group showed tumor regression. Between 2E′ and 2E′/PTX, the latter showed apparently slow tumor growth, resulting in significant difference from D5W control in the tumor size on day 22 post-treatment (FIG. 8b). In the untreated distant tumors, the 2E′ and 2E′/PTX groups showed no tumor growth in 57% and 71% mice, respectively, while 86% of naïve mice grew tumors (FIG. 8c).

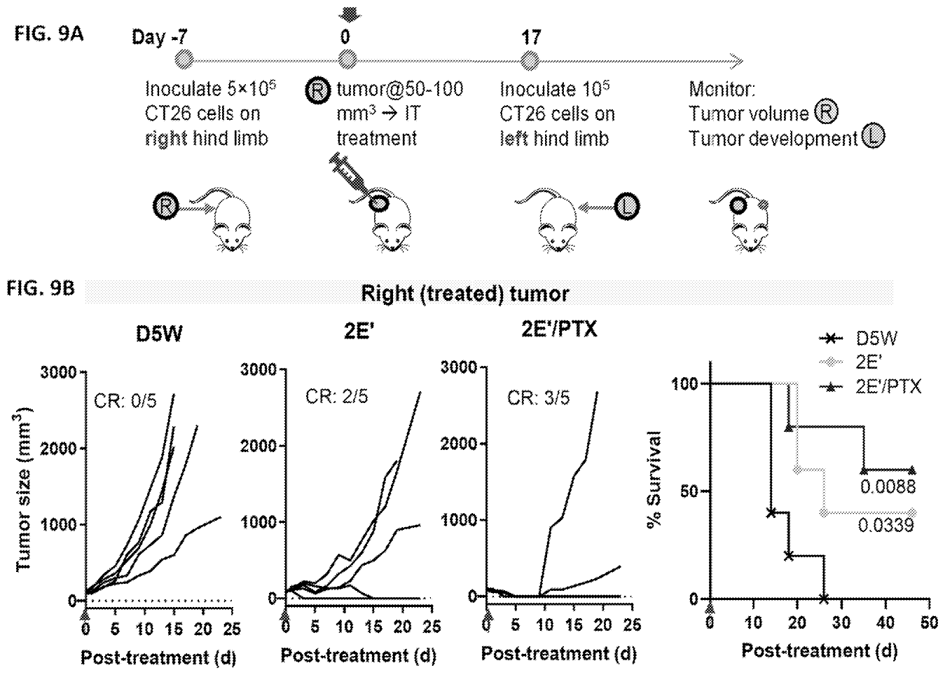

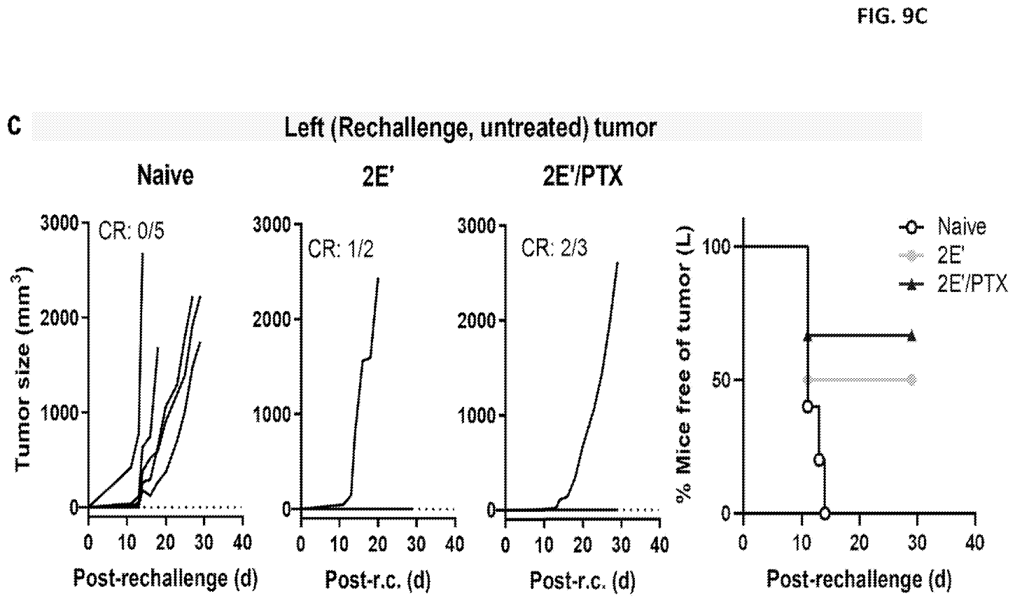

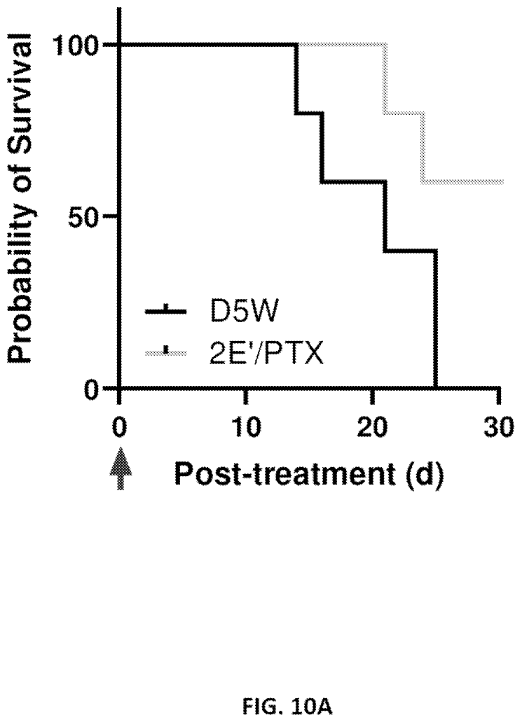

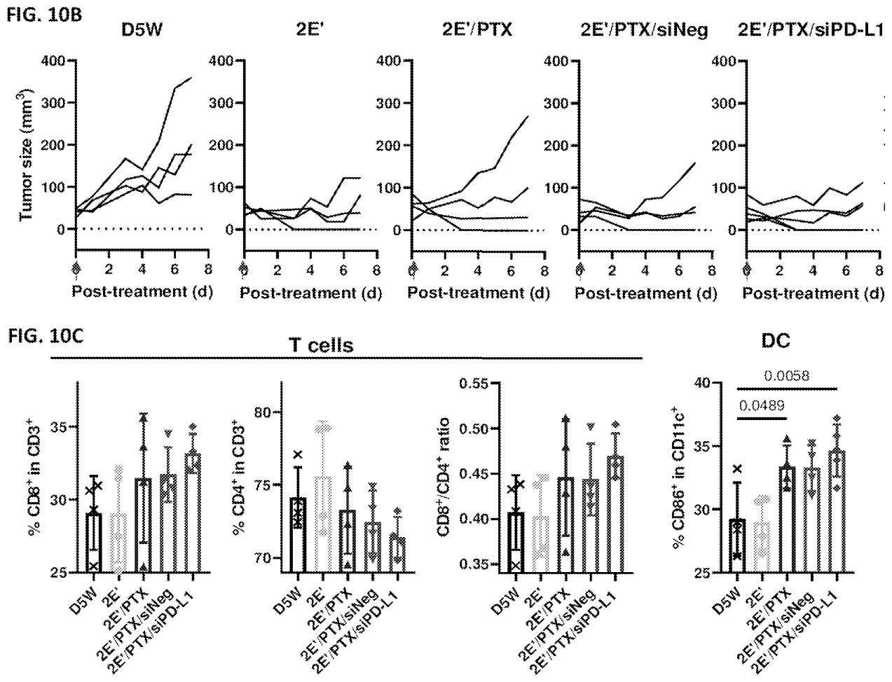

However, the observed CR does not necessarily indicate systemic antitumor immunity, because all four mice in the D5W group also showed no tumor growth, possibly due to the concomitant tumor resistance, a phenomenon describing the primary tumor with a suppressive effect on the secondary tumor³³. Third, we tested whether 2E′/PTX established immunological memory of tumors (FIG. 9a). After a single intratumoral injection, 2E′/PTX (1.4 mg 2E′+0.2 mg PTX per mouse) induced CR of the treated tumors in 60% of animals, and 2E′ in 40% (FIG. 9b). Animals surviving with CR were rechallenged with live CT26 cells on the contralateral side on day 17 after the initial treatment. All the naïve mice grew tumors in 2 weeks, but 2E′ prevented tumor growth in one of the two tumor-free mice and 2E′/PTX in two of the three (FIG. 9c). Finally, 2E′/PTX was tested with orthotopic luciferase-expressing 4T1 (4T1-Luc) breast tumors in Balb/c mice (FIG. 10). The tumor was surgically removed with a residual mass, which was locally treated with D5W or 2E′/PTX (0.2 mg 2E′+0.2 mg PTX per mouse) (FIG. 10a). All five mice in the D5W group showed tumor relapse and lung metastasis on day 17, leaving no surviving animals in 25 days after the treatment (FIG. 10b, 10c). In contrast, two out of five mice in the 2E′/PTX group showed no tumor signal in primary sites and lungs and survived at least for 50 days (duration of observation). These results suggest that 2E′ as an immunostimulant suppressed tumor growth and likely activated antitumor immunity, with additional PTX enhancing the effect.

2E′ Carries PTX and siPD-L1 Simultaneously.

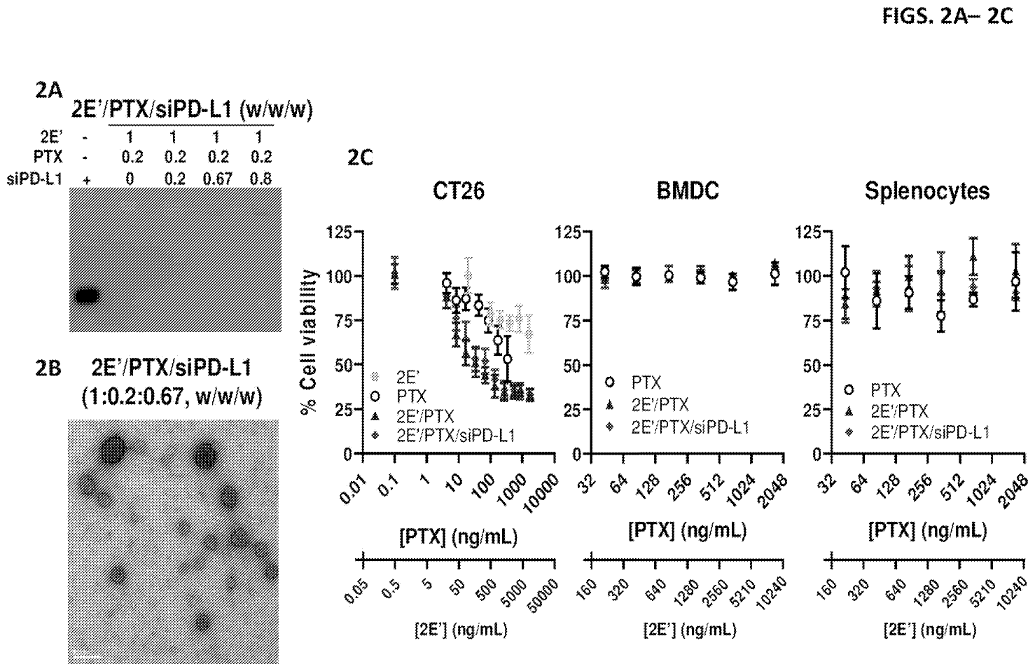

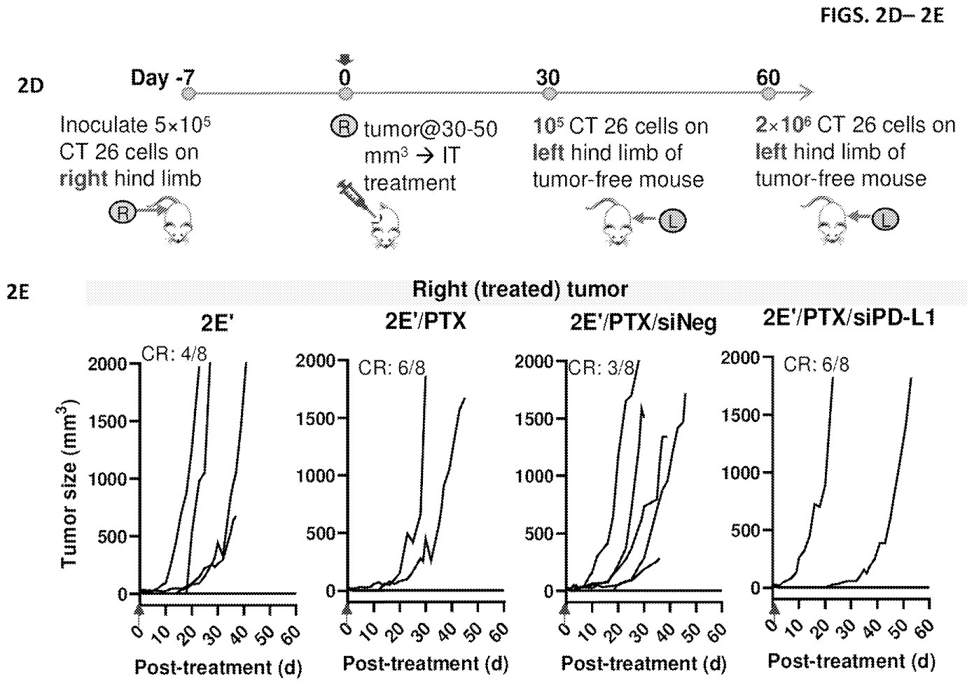

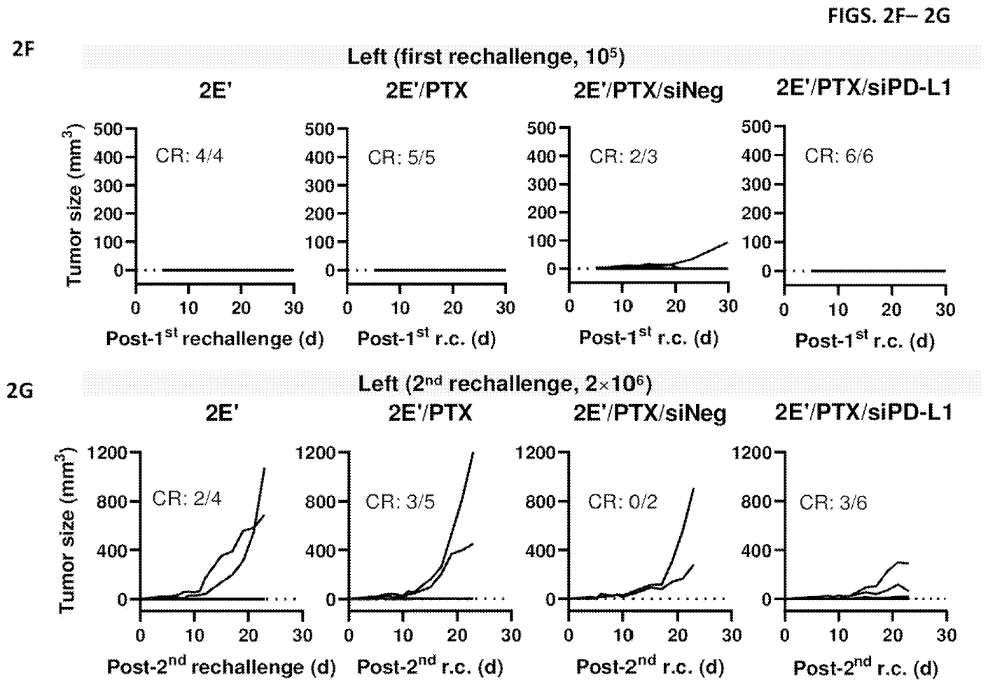

We added siPD-L1 to 2E′/PTX to further enhance antitumor immunity by blocking immune checkpoint interactions. 2E′/PTX/siPD-L1 combination formed a complete complex at a weight ratio of 1:0.2:0.67 (FIG. 2a). The zeta potential of 2E′/PTX/siPD-L1 (+28.9±1.0 mV) was lower than that of 2E′/PTX (+44.6±0.3 mV), reflecting the addition of siPD-L1. The ternary complex showed a spherical shape with an average diameter of 50 nm (FIG. 2b). 2E′/PTX/siPD-L1 appeared bigger than 2E′/PTX (FIG. 1b), suggesting that siPD-L1 formed a complex with multiple 2E′/PTX's. The z-average of 2E′/PTX/siPD-L1 (1/0.2/0.67, w/w/w) (264.4 nm) measured by dynamic light scattering was ˜5 times larger than the TEM measurement (50 nm), indicating that the complexes suspended in aqueous medium undergo some degree of aggregation. 2E′/PTX/siPD-L1 and 2E′/PTX were more toxic to CT26 cells than 2E′ or PTX alone (FIG. 2c), indicating a synergistic effect of 2E′ and PTX (combination index (CI) of <1 for 2E′/PTX and 2E′/PTX/siPD-L1 at all tested levels). However, 2E′/PTX/siPD-L1 as well as PTX or 2E′/PTX did not show significant cytotoxicity to BMDCs and splenocytes at ≤PTX 2 μg/mL (and 2E′ 10 μg/mL) (FIG. 2c). This result is consistent with the differential cell interaction and cytotoxicity of 2E′ (FIG. 1c) and the known resistance of immune cells to PTX³⁴. Despite the sign of siRNA-mediated complexation and mild degree of aggregation in aqueous medium, 2E′/PTX/siPD-L1 entered cancer cells, killed 70% of cancer cells, and inhibited the PD-L1 expression in the surviving IFN-γ-stimulated CT26 and B16F10 cells. 2E′/PTX/siPD-L1 induced the secretion of TNF-α and IL-1β from BMDCs and BMDMs. Replacing siPD-L1 with a control siRNA (siNeg) had a negligible effect on the cytokine secretion except at one concentration, excluding the role of siRNA sequence. 2E′/PTX/siPD-L1 also induced the exposure of CRT on CT26 and B16F10 cells. These results support that 2E′ can carry PTX and siPD-L1 simultaneously, maintaining the activity of each component, to provide selective toxicity to cancer cells, stimulate APCs, and silence PD-L1 expression.

### Single Intratumoral Injection of 2E′/PTX/siPD-L1 Induces Tumor Regression and Protects Animals From Repeated Tumor Challenge in CT26@Balb/c Model.

We tested if a single intratumoral injection of 2E′/PTX/siPD-L1 helped develop antitumor immunity. First, CT26 tumors in Balb/c mice were treated with 2E′, 2E′/PTX. 2E′/PTX/siNeg, or 2E′/PTX/siPD-L1 (FIG. 2d). Consistent with the previous results, 2E′ group showed CR in 50% of mice, and 2E′/PTX in 75%. 2E′/PTX/siPD-L1 also showed CR in 75% of the mice, whereas the 2E′/PTX/siNeg group had 37% tumor-free mice (FIG. 2e). The tumor-free animals were rechallenged with 10⁵ live CT26 cells on the contralateral side on day 30 after treatment. All but one in the 2E′/PTX/siNeg group resisted the rechallenge, indicating that a single treatment of 2E′ and all combinations with PTX helped develop antitumor immunity (FIG. 2f). However, tumor-free mice that had been treated with 2E′/PTX/siPD-L1 performed best when they were rechallenged second time with 20× more live CT26 cells (2×10⁶). At least part of the animals in the 2E′, 2E′/PTX, and 2E′/PTX/siNeg groups showed exponential tumor growth in 10-15 days after the second challenge, but the 2E′/PTX/siPD-L1-treated mice either did not grow tumor (CR: 50%) or attenuated the growth compared to the others (FIG. 2g). These results demonstrate that all tested complexes developed antitumor immune memory; among them, 2E′/PTX/siPD-L1 showed the strongest protective immunity, enough to inhibit tumor growth in the surviving mice receiving multiple rechallenges of live tumor cells. 2E′/PTX/siNeg was apparently inferior to 2E′/PTX in primary tumor control and resistance to rechallenges. We speculate that siNeg only diminished the positive charge of 2E′, which is critical to the immunostimulatory effect³⁵⁻³⁷, without a functional activity. In a repeated study, we examined the tumor immunoenvironment on day 7 after the treatment, the optimal time frame to observe DC maturation and T cell expansion in response to the treatment³⁸. Since CT26 tumors receiving the treatments were too small to analyze at this time, we analyzed tumor-draining lymph nodes (TDLNs). 2E′/PTX, 2E′/PTX/siNeg, and 2E′/PTX/siPD-L1-treated groups showed apparent increase in CD8⁺ T cells and mature DCs and decrease in CD4⁺ T cells compared to the D5W-treated control (FIGS. 10b/10c). These results are consistent with the immunostimulatory effects of 2E′ (FIG. 1) and PTX (FIG. 10b/10c) as well as antitumor immune memory seen in the previous study (FIG. 2f, 2g).

### Single Intratumoral Injection of 2E′/PTX/siPD-L1 Induces and Maintains Immunoactive Phenotype in Poorly Immunogenic B16F10 Tumors.

We used the poorly immunogenic B16F10 melanoma model to examine the

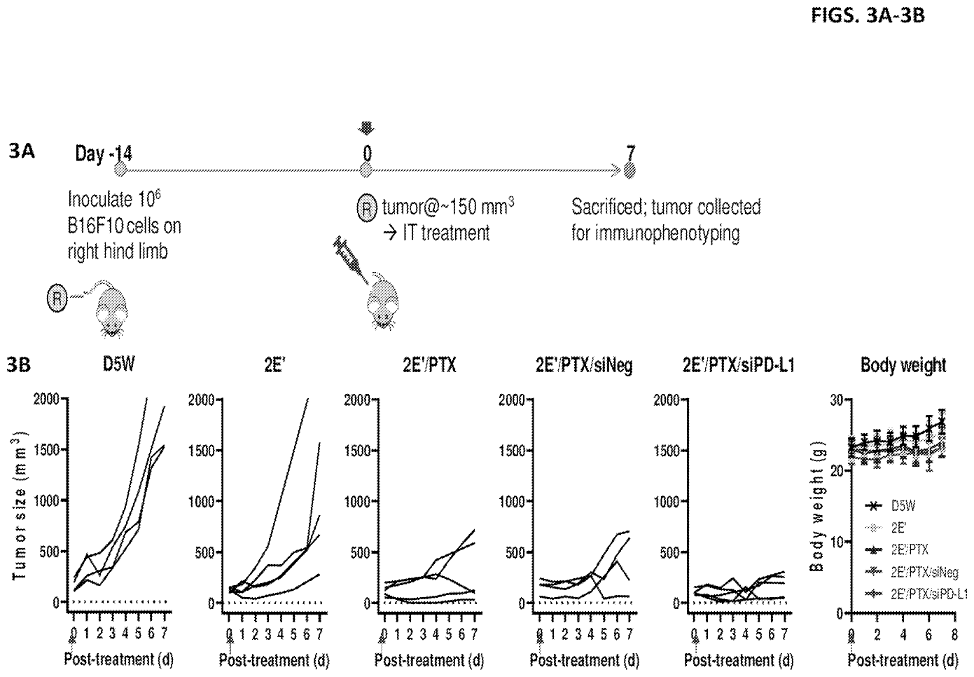

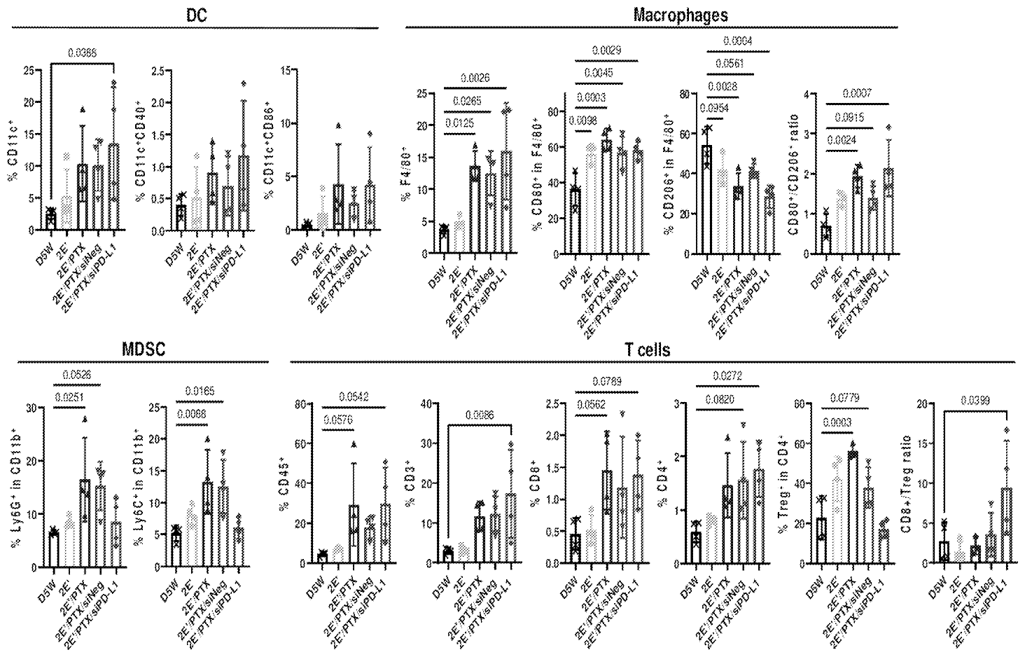

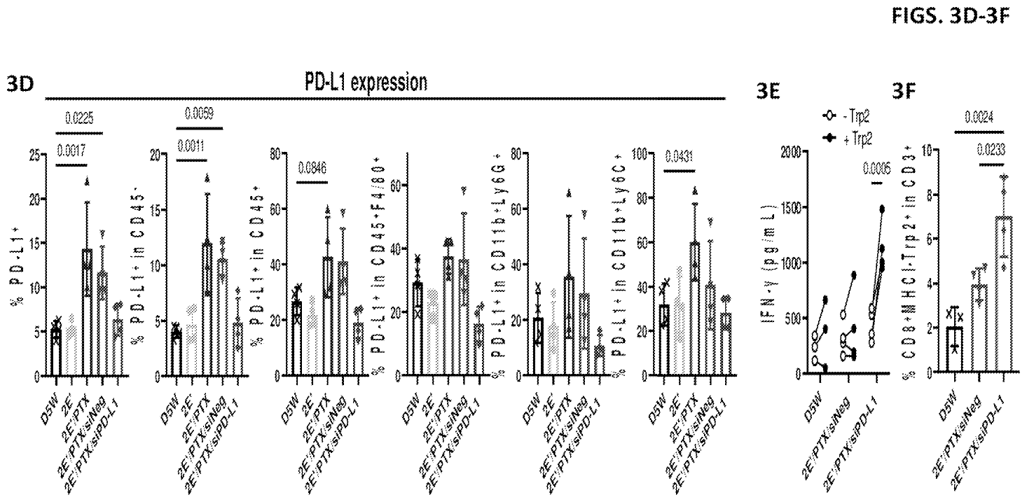

immunophenotype of the treated tumors. Treatments were given when tumors grew to ˜150 mm³ (at least three times larger than CT26 tumors) (FIG. 3a) to afford a sufficient size for analysis. Again, 2E′/PTX/siPD-L1-treated group was most effective (FIG. 3b). Tumors of the D5W group already approached the endpoint 7 days post-treatment, whereas those of the 2E′/PTX/siPD-L1-treated group barely grew or shrank during this time. None of the treated mice showed significant changes in the body weight (FIG. 3b) or lesions in parenchymal organs, suggesting the safety of locally administered complexes in 7 days.

Tumors collected 7 days after treatment were analyzed by flow cytometry (FIG. 3) and immunohistochemistry (IHC). The tumors treated with 2E′/PTX, 2E′/PTX/siNeg, or 2E′/PTX/siPD-L1 had apparently more mature DCs and macrophages, skewed to pro-inflammatory CD80⁺ M1-like phenotype, compared to those treated with D5W (FIG. 3c). Interestingly, 2E′/PTX and 2E′/PTX/siNeg increased the percentage of myeloid-derived suppressor cells (MDSCs) in tumors as compared to the D5W group. However, the 2E′/PTX/siPD-L1 treated group showed no increase in the MDSC fraction. 2E′/PTX, 2E′/PTX/siNeg, and 2E′/PTX/siPD-L1 increased CD8⁺ T cell infiltration into tumors (FIG. 3c). CD4⁺ T cells similarly increased in these groups. However, the 2E′/PTX/siPD-L1-treated tumors had the least fraction of regulatory T cells (Treg) in CD4⁺ T cells, showing a significantly higher CD8⁺/Treg ratio than the D5W control group. These results suggest that 2E′/PTX/siPD-L1 maintained the most immunoactive phenotype, resisting the influx of immunosuppressive cells. We also determined the PD-L1 expression in the tumor microenvironment (FIG. 3d). 2E′/PTX or 2E′/PTX/siNeg-treated tumors showed significantly higher PD-L1 expression than D5W-treated tumors. Both tumor cells and immune cells, including macrophages and MDSCs, showed a trend of increased PD-L1 expression, which suggests negative feedback to 2E′/PTX-mediated immunostimulation. It is reported that PD-L1 expression induces the conversion of immature myeloid cells to immunosuppressive MDSCs³⁹ and enhances the differentiation and function of Treg⁴⁰. The increased PD-L1 expression in 2E′/PTX and 2E′/PTX/siNeg-treated tumors is consistent with the increased populations of MDSCs and Treg (FIG. 3c). In contrast, 2E′/PTX/siPD-L1 prevented the overexpression of PD-L1 (FIG. 3d). Therefore, the ability of 2E′/PTX/siPD-L1-treated group to maintain relatively low level of MDSCs and Treg may be ascribed to the silencing effect of siPD-L1. The PD-L1 expression profiles and immune cell populations collectively indicate that 2E′/PTX/siPD-L1 not only induced an immunostimulatory TME (based on 2E′ and PTX) but also maintained one (based on siPD-L1). RNA sequencing showed a consistent trend as the tumor growth and immunophenotyping results (FIG. 3). 2E′/PTX/siNeg and 2E′/PTX/siPD-L1 treatment suppressed the expression of tumor-supporting cytokines, such as IL-6, LIF, IL-10, TNF, IL-1a, compared to the D5W control. RNA sequencing also supported the elevated level of Cd274, the PD-L1 encoding gene, in 2E′/PTX/siNeg-treated tumors but not in 2E′/PTX/siPD-L1-treated ones.

### Single Intratumoral Injection of 2E′/PTX/siPD-L1 Primes Systemic Antitumor Immunity and Immune Memory in B16F10 Tumors.

To test if the activation of local antitumor immunity translated to systemic antitumor immunity, we collected splenocytes of the B16F10 tumor-bearing mice 7 days after treatment and stimulated them with Trp2 peptide, a B16F10 melanoma-associated antigen⁴¹, to measure IFN-γ production (FIG. 3c). Splenocytes of the 2E′/PTX/siPD-L1-treated mice produced more IFN-γ than the D5W- or 2E′/PTX/siNeg-treated groups, indicating the activation of the systemic antitumor immunity. To test if this activation involved CD8⁺ T cells, we quantified CD8⁺ T cells binding to MHCI-Trp2 peptide tetramer complex. 2E′/PTX/siPD-L1 induced significant increase of Trp2⁺ CD8⁺ T cells compared to D5W or 2E′/PTX/siNeg (FIG. 3f), supporting the tumor-specific T cell response.

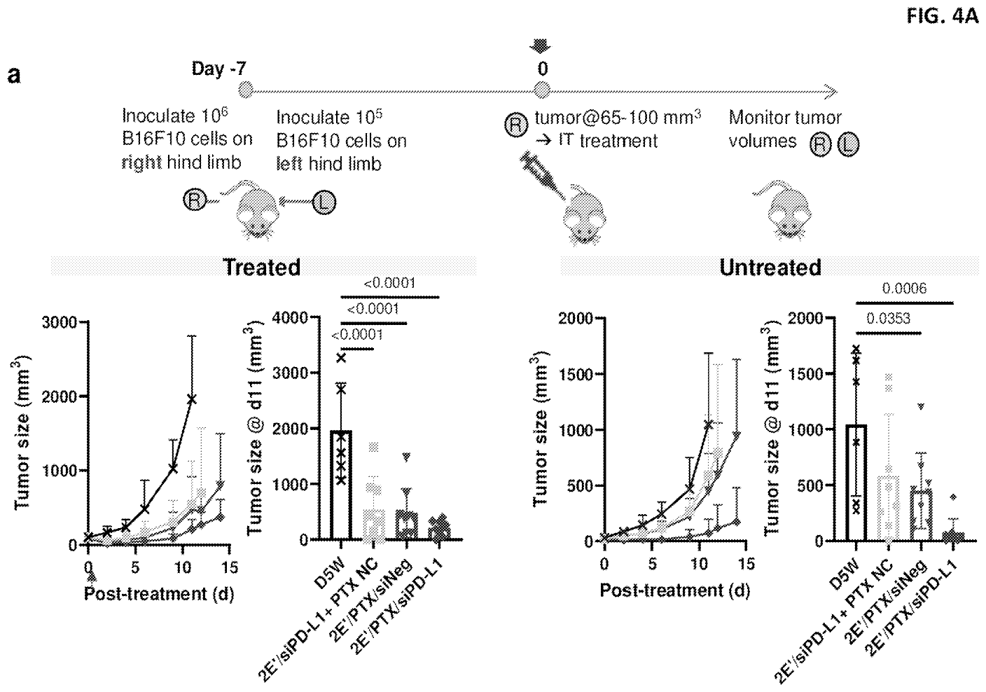

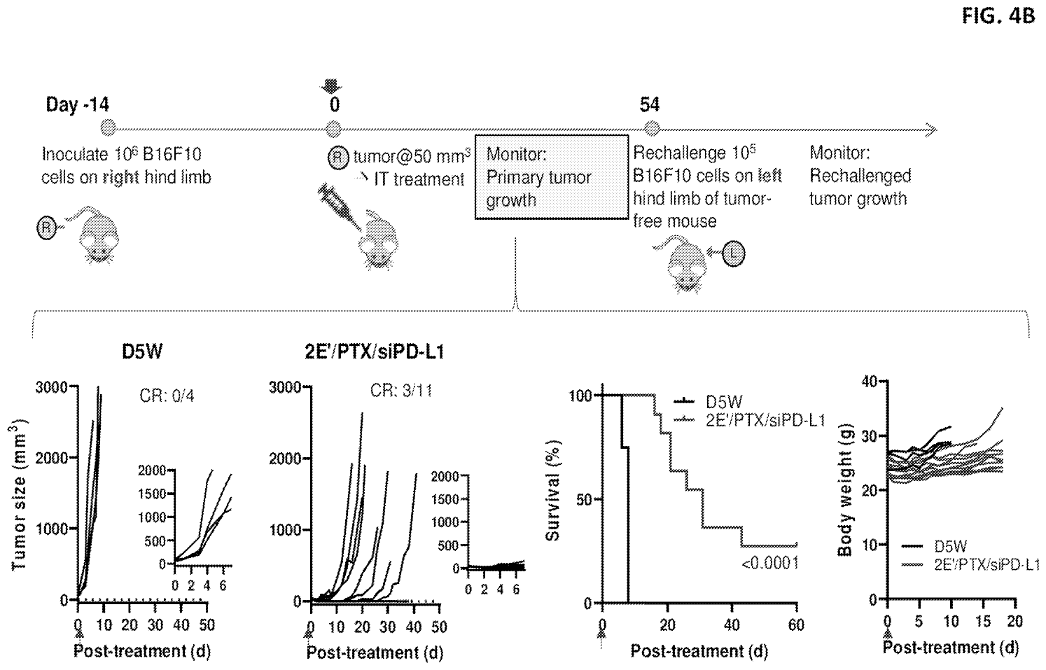

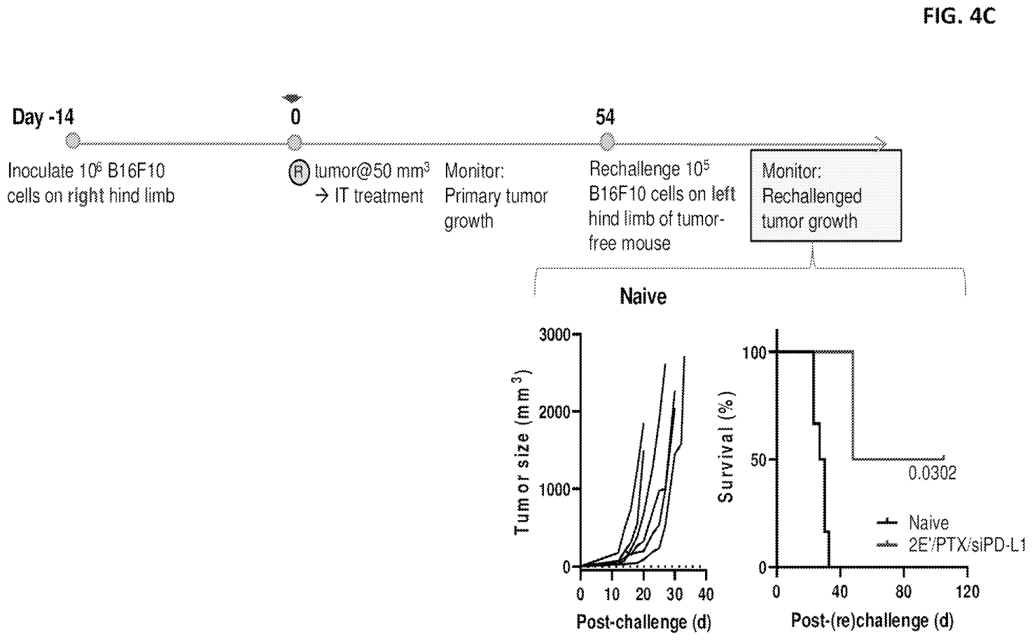

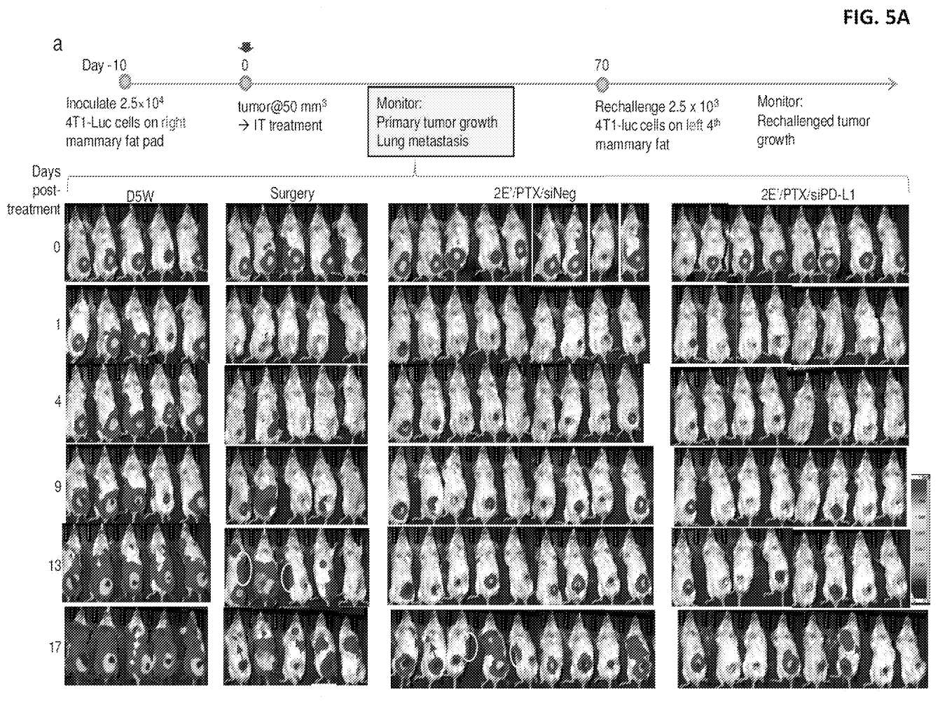

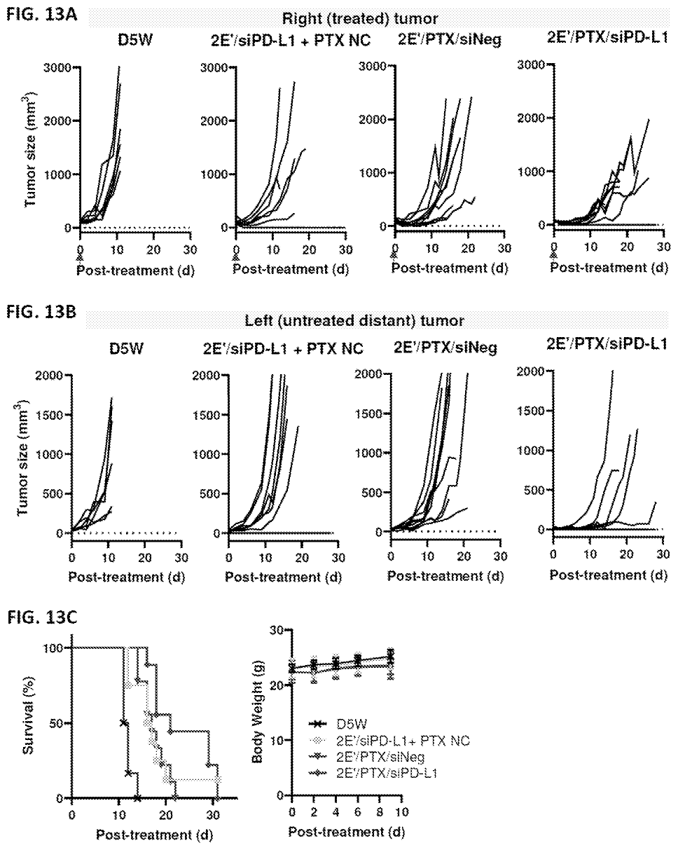

Based on the evidence of systemic antitumor immunity, we tested whether 2E′/PTX/siPD-L1 would have an abscopal effect on untreated distant tumors in a bilateral B16F10 tumors in C57BL/6 mice, comparing with D5W and 2E′/PTX/siNeg (FIG. 4a, FIG. 13). To exclude the possibility of systemically absorbed PTX playing a role, a mixture of 2E′/siPD-L1 and PTX was also compared (due to the low water solubility, PTX was included in the mixture as nanocrystals^{42,43}, which was superior to the commercial PTX formulation (Abraxane) in B16F10 model⁴³). Consistent with systemic activation of tumor antigen-specific T cells (FIG. 3c, 3f), 2E′/PTX/siPD-L1 delayed tumor growth in both treated and untreated distant tumors better than 2E′/PTX/siNeg and the 2E′/siPD-L1+PTX mixture, yielding the smallest tumor size in both sides on day 11 post-treatment (the last time point all groups were surviving). The comparison of 2E′/PTX/siPD-L1 and 2E′/siPD-L1+PTX mixture suggests that the untreated distant tumor is better suppressed when PTX was encapsulated in 2E′ (2E′/PTX/siPD-L1): i.e., the effect on distant tumor would be the consequence of a local event, where the retention of PTX is beneficial, rather than a function of systemically absorbed PTX.

We then asked if 2E′/PTX/siPD-L1 treatment would generate antitumor immune memory in B16F10 tumors (FIGS. 4b, 4c). We treated B16F10-bearing mice with D5W and 2E′/PTX/siPD-L1 and confirmed the reproducible initial tumor growth pattern (FIG. 3). All mice in the D5W group reached the endpoint in 8 days after the treatment. However, 2E′/PTX/siPD-L1-treated animals showed a median survival time of 31 days from the treatment, inducing CR in 27% of the mice. The surviving tumor-free mice were rechallenged with 10⁵ live B16F10 cells on the contralateral side on day 54 after the treatment. All age-matched naïve mice developed tumors in 14 days and reached the endpoint in 33 days after inoculation. Of the three tumor-free mice in the 2E′/PTX/siPD-L1 treated group, one died for an unknown reason on day 16 after rechallenge without growing tumor; the other showed tumor on day 18 after rechallenge, which grew to the endpoint in 48 days; and the third did not develop tumor in 105 days (duration of observation) after the rechallenge. The delayed or no growth of rechallenged tumors suggests that the local treatment with 2E′/PTX/siPD-L1 establish immune memory of B16F10 tumors.

### Intratumoral Injection of 2E′/PTX/siPD-L1 Inhibits Growth and Metastasis of 4T1 Tumors.

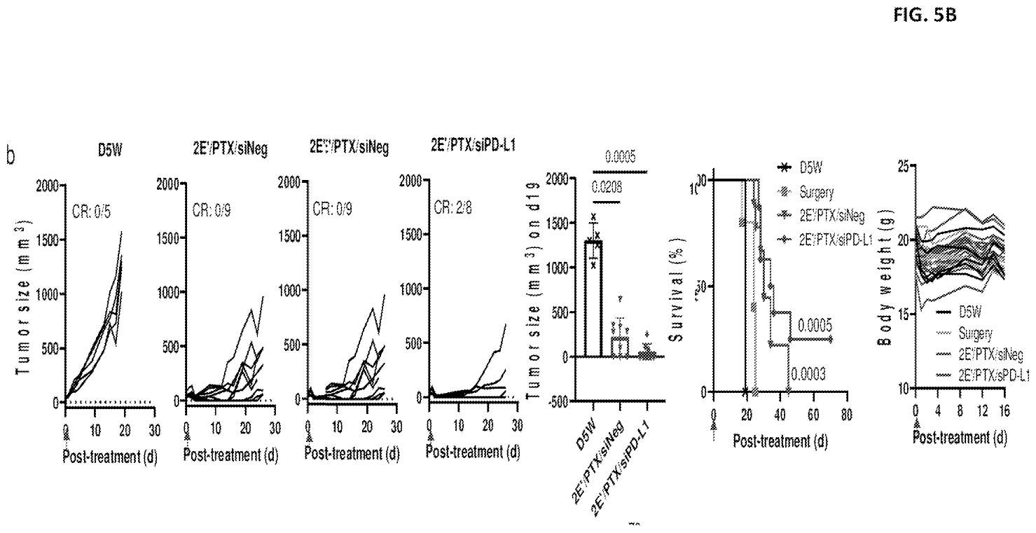

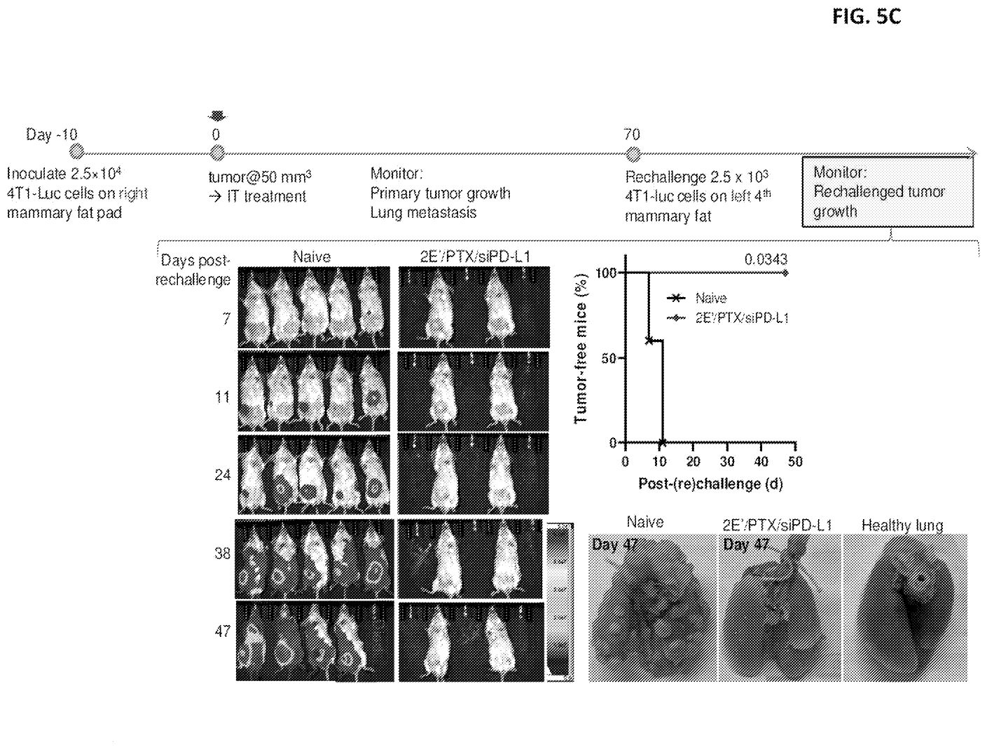

To evaluate broad utility of 2E′/PTX/siPD-L1, we tested it in orthotopic 4T1-Luc tumors, another poorly immunogenic tumor model^{44,45}, which spontaneously metastasizes to multiple distant sites including lymph nodes, blood, lung, liver, brain and bone while primary tumor is present as human breast tumors do⁴⁶, comparing with three control groups (D5W, 2E′/PTX/siNeg, and complete surgical resection to mimic a clinical scenario) (FIG. 5). In the D5W group, tumors grew exponentially, and all animals reached physiological endpoints in 19 days with widespread metastasis. In the surgery group, 80% showed tumor relapse in the surgical site in 13 days from the surgery, and all showed lung metastasis in 17 days reaching the endpoint in 25 days. In contrast, 2E′/PTX/siNeg and 2E′/PTX/siPD-L1-treated groups showed immediate loss of luminescence signal (in 1 day) and shrinkage of tumors (in 2 days) following treatment, although most of them regrew at later times. The occurrence of lung metastasis was also reduced by these treatments, with 55% in the 2E′/PTX/siNeg group and 88% in the 2E′/PTX/siPD-L1 group showing no luminescence signals in the lungs by 17 days after treatment. The median survival times were 30 days (2E′/PTX/siNeg) and 35 days (2E′/PTX/siPD-L1). The 2E′/PTX/siNeg-treated group showed no CR, whereas the 2E′/PTX/siPD-L1 group had 25% CR. The two tumor-free mice in the 2E′/PTX/siPD-L1 group were rechallenged with 4T1-Luc cells on the contralateral side. Neither grew tumors in 47 days from the rechallenge (duration of observation), whereas all of the age-matched naïve mice did in 11 days (FIG. 5c). These results demonstrate that a single treatment of 2E′/PTX/siPD-L1 outperformed the surgical removal, a clinical benchmark treatment, in the management of both primary tumors and metastasis, establishing antitumor immune memory in surviving animals.

### 2E′/PTX Combined With Cyclic Dinucleotide (CDN), a STING Agonist, Eliminates Established Tumors and Develops Antitumor Immunity in CT26@Balb/c Model.

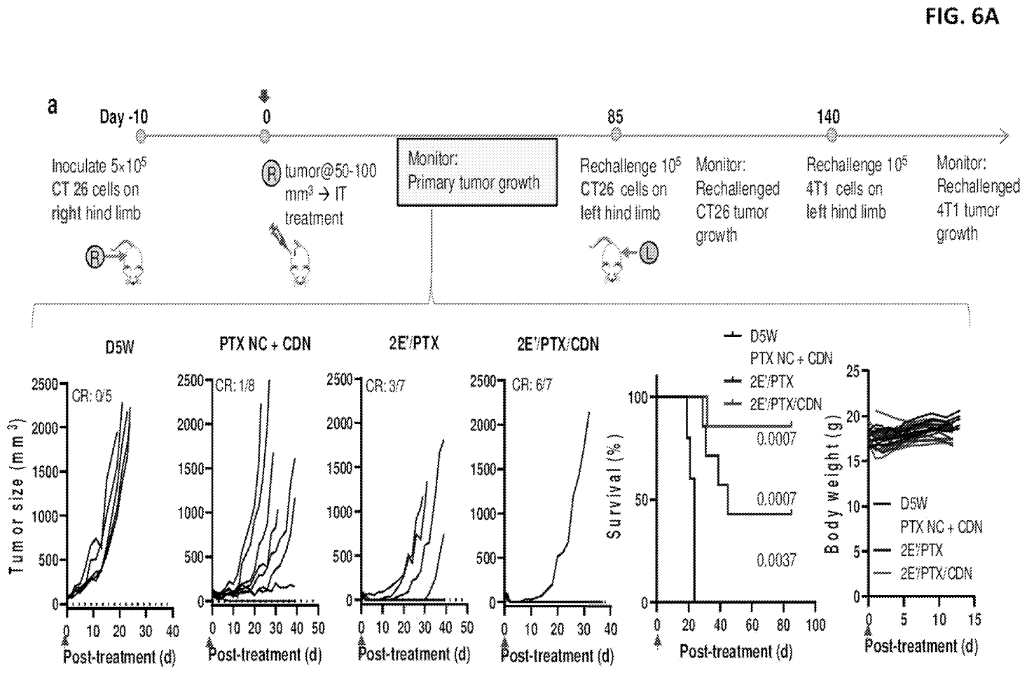

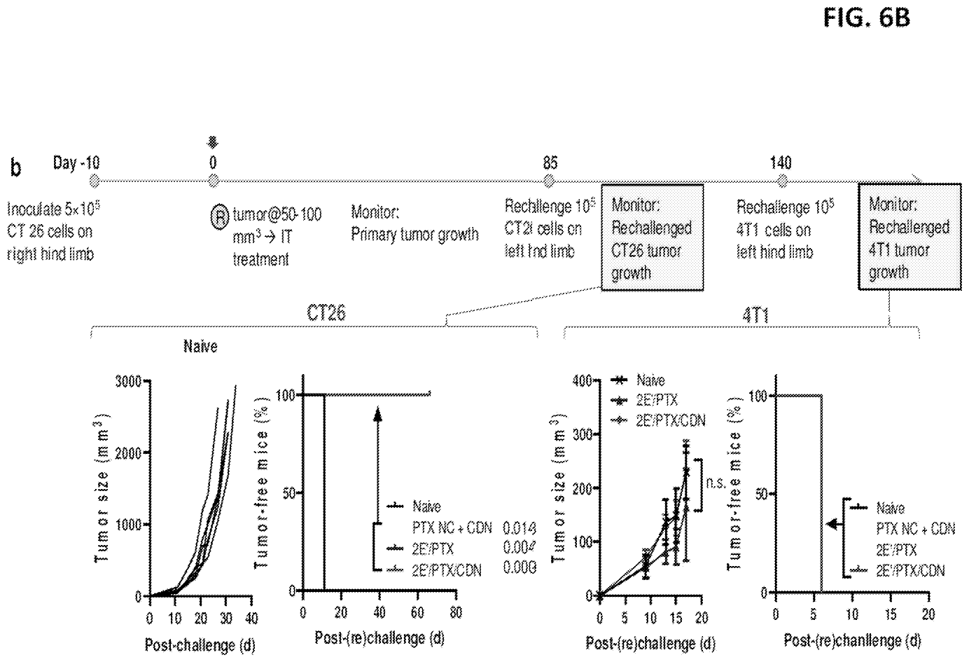

To test the applicability of 2E′ to carrying other drugs, we replaced siPD-L1 with CDN (a bisphosphorothioate analog of 2′3′-c-di-AMP), an agonist of the stimulator of interferon genes (STING) pathway⁴⁷, and tested it in the CT26 model, along with three control groups (D5W. PTX nanocrystal+CDN mixture, 2E′/PTX) (FIG. 6). The PTX+CDN mixture delayed tumor growth compared to the D5W control, extending the median survival time from 24 to 35 days and achieving 13% CR. 2E′/PTX was more effective than the PTX+CDN mixture, with a median survival time of 45 days and a CR rate of 43%. 2E′/PTX/CDN showed the best antitumor effects: 86% of the treated mice survived tumor-free after a single treatment. When rechallenged with live CT26 cells, none of the tumor-free mice grew the rechallenged tumor for 66 days (duration of observation), while all the age-matched naïve mice did in 11 days and died in 34 days. These results indicate that the single intratumoral treatment of 2E′/PTX/CDN induced strong antitumor immune responses, which eradicated the established tumors and protected the cured animals from the reinvasion of tumors. The surviving animals with 2E′/PTX or 2E′/PTX/CDN treatment did not reject unrelated syngeneic 4T1 tumors (FIG. 6b), indicating that the antitumor immunity was tumor-specific.

### Discussion

Immunotherapy has shown dramatic outcomes in cancer therapy, but the current ceiling of response rates indicates the need for improved approaches. Here we show that an immunoactive nanoparticle, 2E′ carrying PTX and siPD-L1 or CDN, induced immediate regression of established tumors upon a single local administration and enhanced the activation of antitumor immunity, leading to systemic, long-term protection of surviving animals. Each component played distinct roles to activate antitumor immunity, with 2E′ stimulating innate immunity against tumor due to the immunoadjuvant effect (FIG. 1d-1j) and PTX and 2E′ inducing ICD (i.c., in situ generation of tumor antigens) (FIG. 2c), which then activates T-cell immunity against tumors. siPD-L1 contributed to the later step by preventing tumor expression of PD-L1 that would otherwise engage in immune checkpoint interaction and MDSC and Treg recruitment (FIG. 3c-f). CDN leveraged 2E′ and PTX in activating innate immune responses⁴⁸. These ternary complexes were superior or comparable to the recently reported local immunotherapeutics in similar tumor models^{19,49}. For example, the 2E′/PTX/siPD-L1 (FIG. 4, 5) compares favorably with the microfabricated polylactic-co-glycolic acid particles carrying a STING agonist⁴⁹ in the median survival time from the treatment (31 d vs. 21 d⁴⁹ in B16F10 model and 35 d vs. 24 d⁴⁹ in 4T1 model) and % CR (27% vs. 0%⁴⁹ in B16F10 model and 25% vs. 0%⁴⁹ in 4T1 model) after a single intratumoral administration. 2E′/PTX/siPD-L1 (FIGS. 2 and 5) and 2E′/PTX/CDN (FIG. 6) are also comparable to the camptothecin prodrug-based hydrogel carrying a STING agonist, which induced the regression of established CT26 tumors and retarded the growth and metastasis of 4T1 tumors after a single intratumoral injection¹⁹, with the additional advantage of broad applicability. Moreover, 2E′/PTX/CDN contained 20 μg CDN, ½ or ⅓ of the doses in the literature (4×10 μg⁴⁹, 1×40 μg⁴⁹, or 3×20 μg⁵⁰) but achieved 86% CR and tumor-specific immunity after a single administration.

2E′ has several outstanding features that make it uniquely suitable for local

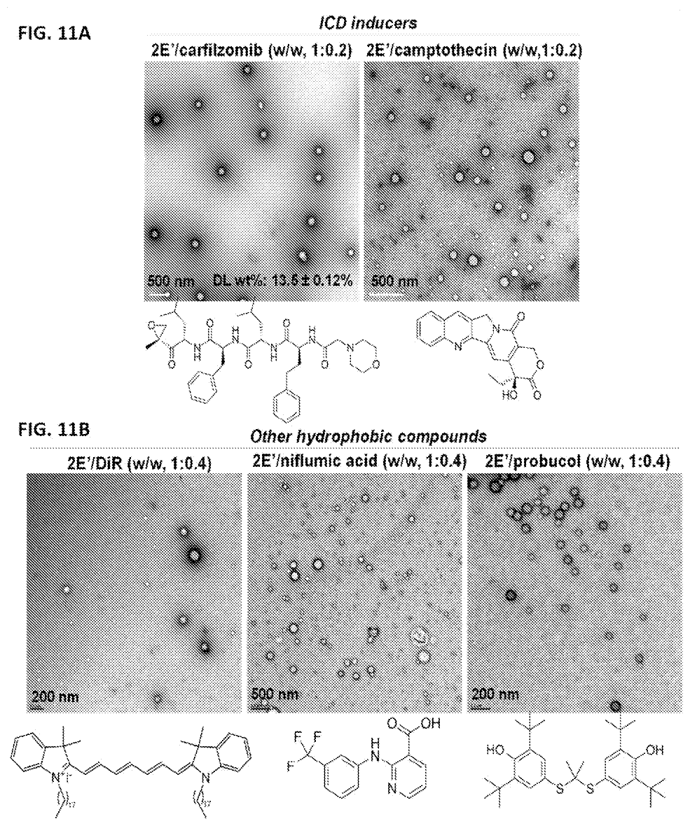

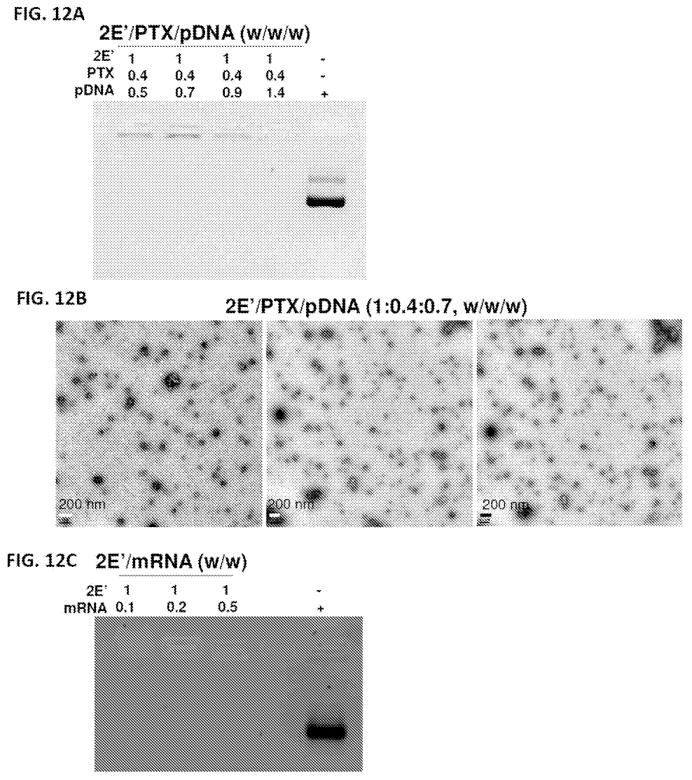

immunotherapy of cancer. First, the carrier itself is immunoactive, attributable to the inherent properties of the parent polymer PEI^{17,21} as well as the conjugated LCA²². The nanoparticle formation by self-assembly of the amphiphilic PEI derivative (FIG. 1b) may also have enhanced the interaction of the polymer with APCs. Second, 2E′ has selective toxicity against cancer cells as compared to immune cells (BMDCs and splenocytes), causing an ICD phenotype (CRT exposure) in the affected cancer cells (FIG. 1g). Cancer cells undergo an unusual glycolytic pathway for energy production, thereby secreting a large quantity of lactic acid, which makes the plasma membrane more negatively charged than normal cells²⁴. The dense negative charge may have facilitated the interaction with 2E′, accounting for the selective toxicity of 2E′ to CT26 and B16F10 cells relative to BMDCs. Based on immunoactivity and toxicity to cancer cells, 2E′ alone attenuated or suppressed the treated tumor. However, the incorporation of PTX, siPD-L1, or CDN further increased the antitumor effect and helped establish strong antitumor immune memory (FIGS. 2-6). Therefore, the third feature of 2E′ is its compatibility with both hydrophobic anticancer drugs and negatively charged nucleic acids or nucleotides, which bring complementary functions but are difficult to co-deliver without a carrier. The combination of 2E′, PTX, and siPDL-1 yields favorable outcomes, including the synergistic effects of 2E′ and PTX (FIG. 2c) and intracellular delivery of siPD-L1. We envision that 2E′ would be suitable for the delivery of diverse combinations of immunoactive agents. 2E′ carries PTX and siPD-L1 via the hydrophobic LCA core and cationic PEI backbone, respectively; therefore, it is possible to replace PTX with other hydrophobic ICD inducers (FIG. 11) and siPD-L1 with different siRNAs, nucleotides such as CDN (FIG. 6), or other nucleic acids (FIG. 12). The modularity of 2E′ and versatile functions of nucleic acid therapeutics would allow to develop comprehensive combinations according to the type of tumors without needing a redesign of carriers or drugs. These features of 2E′ distinguish this study from others that used a similar PEI-LCA conjugate as a simple drug carrier⁵¹ or combined PTX and siPD-L1 in a different carrier¹⁸.

In summary, we have developed 2E′, an immunostimulatory carrier of hydrophobic drugs and nucleic acids/nucleotides. 2E′ and its combination with PTX and siPD-L1 or CDN induced potent antitumor immunity by a single local administration, causing immediate regression of large established tumors and tumor-free survival in multiple tumor models. The treated animals showed immunoactive phenotype in TME and tumor-specific T cell responses and resisted metastasis or rechallenge, indicating the induction of systemic antitumor immunity and immune memory. This study supports that effective in-situ induction of antitumor immunity can lead to the systemic protection from distant and recurrent diseases; 2E′ provides a simple and versatile platform for local immunotherapy by accommodating combinations of chemotherapeutic drugs and nucleic acids that address multiple events involved in the antitumor immunity.

### Materials and Methods

### Materials

Linear polyethylenimine base form (PEI base form, MW: 2.5 kDa) and polyethylenimine

hydrochloride (PEI salt form, MW 4 kDa equivalent to 2.5 kDa PEI base form) were purchased from Polysciences, Inc. (Warrington, PA). 1,1′-carbonyldiimidazole (CDI), lithocholic acid (LCA, ≥97%), and EtBr (10 mg/mL) were purchased from Sigma-Aldrich (St. Louis, MO). D-Luciferin potassium salt was purchased from Gold Biotechnology (St. Louis, MO, USA). siRNA specific for the mouse pdcd1lg1 mRNA, sense, 5′-CCCACAUAAAAAACAGUUGTT-3′, SEQ ID NO:1; antisense, 5′-CAACUGUUUUUUAUGUGGGTT-3′, SEQ ID NO:2; negative siRNA (sense, 5′-UGAAGUUGCACUUGAAGUCdTdT-3′, SEQ ID NO:3; antisense, 5′-GACUUCAAGUGCAACUUCAdTdT-3′, SEQ ID NO:4) and Cy5-labeled negative siRNA were purchased from IDT (Coralville, Iowa, USA). iTAg Tetramer/APC—H-2 Kb TRP2 (SVYDFFVWL) was purchased from MBL International Corporation (Woburn, MA). Cyclic dinucleotide (CDN)-2′3′-c-di-AM(PS)2 (Rp,Rp) was purchased from InvotroGen (San Diego, CA). Firefly luciferase-expressing plasmid DNA (pLuc) were replicated in DH5-α competent Escherichia coli as reported previously⁵². CleanCap® EGFP mRNA (mRNA) was purchased from TriLink BioTechnologies (San Diego, CA).

### 2E′ Synthesis and Characterization

2E′ was synthesized as described in the previous report²³. Briefly, 8.7 mg (50 μmol) of CDI and 15.8 mg of LCA (40 μmol) were dissolved in 2.7 mL of chloroform under stirring. After 1 h, the mixture was slowly added to 10 mL chloroform solution containing 50 mg of PEI base (2.5 kDa) (20 μmol) at 60° C. and reacted for 24 h under stirring. The LCA-PEI conjugate (2E′) was purified by dialysis (molecular weight cut-off (MWCO): 1000 Da) against 95% ethanol, followed by acidified deionized (DI) water. For fluorescence labeling of 2E′ or PEI, 20 mg of 2E′ or PEI was dispersed in anhydrous ethanol (0.5 mL) containing sulfo-cy5-NHS (1 mg). The reaction solution was stirred in dark for 24 h and dialyzed against DI water using a dialysis bag with a molecular weight cut off of 1 kDa. 2E′ and PEI base were dissolved in DMSO and analyzed by a Bruker DRX500-2 NMR spectrometer equipped with a BBFO probe.

### Measurement of the Critical Assembly Concentration (CAC) of 2E′

2E′ was dissolved in DI water to 2 mg/mL and diluted stepwise by DI water to 1.5×10⁻⁵ mg/mL. To 0.5 mL of each solution, 5 μL of Nile red solution in acetone (2 mg/mL) was added to record the emission spectrum at 636 nm by the excitation at 552 nm.

### Cellular Interaction of 2E′

CT26 cells and splenocytes obtained from female Balb/c mice were seeded in Nunc™

glass bottom dishes (Thermo Scientific) at a density of 3×10⁵ and incubated for 1 h. The cells were rinsed, fixed in 4% paraformaldehyde, stained with 5 μg/mL Wheat Germ Agglutinin-Alexa Fluor™ 647 Conjugate for 10 min, and imaged by the Nikon A1R confocal microscope.

### Preparation and Characterization of 2E′/PTX and 2E′/PTX/siPD-L1

To prepare binary complexes of 2E′ and PTX (2E′/PTX), 2E′ and PTX were mixed at different weight ratios in a chloroform/ethanol mixture (3:1, v/v) in a round-bottom flask, dried by rotary evaporation to form a thin film on the wall of the flask, and hydrated in DI water by bath sonication for 5 min. 2E′/siPDL-1 binary complex was prepared by co-incubation of 2E′ and siPD-L1 in nuclease-free water varying the weight ratios for 30 min at room temperature. A ternary complex of 2E′, PTX, and siPD-L1 (2E′/PTX/siPD-L1) was prepared by incubating 2E′/PTX with siRNA for 30 min at room temperature. The formation of the ternary complex was confirmed by 1.1% agarose gel electrophoresis. The size and zeta potentials of all complexes were determined by the Malvern Zetasizer Nano ZS90 (Worcestershire, UK). Their morphology was examined by the FEI Tecnai T20 transmission electron microscope (Hillsboro, OR) after negative staining with 1% uranyl acetate.

In Vitro Release of PTX from 2E′/PTX

One milliliter of 2E′/PTX (2.5:1, w/w) or 2E′/PTX (5:1, w/w) equivalent to 200 μg of PTX was put in a dialysis cassette (MWCO: 10 kDa), placed in 15 mL of 0.2% Tween 80 aqueous solution, and incubated at 25° ° C. under constant agitation. At timed intervals, the entire release medium was replaced with fresh medium. The amount of PTX in the sampled medium at each time point was analyzed by the Agilent 1100 HPLC system (Palo Alto, CA) equipped with Ascentis C18 column (25 cm× 4.6 mm, particle size 5 mm). The mobile phase was composed of water and acetonitrile (50:50) and run at 1 mL/min. PTX was detected by a UV detector at wavelength of 227 nm.

### CRT Expression in Tumor Cells

2×10⁵ CT26 or B16F10 cells were seeded in a 6-well plate overnight. The cell culture medium was replaced with a medium containing 2E′, PEI, PTX, 2E′/PTX or 2E′/PTX/siPD-L1 at varying concentrations. After 6 h or 24 h, the cells were collected, resuspended in staining buffer, incubated with anti-mouse CD16/32 antibody to block non-specific binding of the immunoglobulin to Fc receptors, and stained with Alexa Fluor 488-conjugated anti-CRT monoclonal antibody (ab196158, Abcam). The cells were incubated with 0.5 μg/mL propodium iodide for 1 min prior to the analysis with the BD Accuri C6 Flow Cytometer. For visualizing CRT exposure, 2×10⁵ CT26 or B16F10 cells were seeded in a confocal dish, incubated with different treatments for 24 h, washed, stained in the same manner as above, and fixed with 4% paraformaldehyde. Confocal images of the fixed cells were taken with the Nikon A1R confocal microscope (Nikon America Inc., Melville, NY) after brief staining with 2 μg/mL Hoechst 33342.

### BMDC and BMDM Differentiation

Bone marrow-derived dendritic cells (BMDCs) and bone marrow-derived macrophages (BMDMs) were differentiated from mouse bone marrow cells. The bone marrow was collected from the femur of female Balb/c or male C57BL/6 mice (7 weeks old), pipetted several times, and passed through a 100 and 40 μm cell strainer to obtain single-cell suspension. The cells were collected by centrifugation at 500 ref for 8 min, treated with ACK lysis buffer, rinsed, and cultured in Alpha minimum essential medium (MEM-Alpha, ribonucleosides, deoxyribonucleosides, 4 mM L-glutamine, 1 mM sodium pyruvate) supplemented with 100 units/mL penicillin, 100 μg/mL streptomycin, 10 mM β-mercaptoethanol, 20 ng/ml murine GM-CSF and 20% fetal bovine serum. After 7 days, floating or loosely adherent cells were collected by centrifugation and identified as BMDC by APC anti-mouse CD11c antibody labeling. Adherent cells were identified as BMDM by FITC anti-mouse F4/80 antibody labeling.

### BMDC and BMDM Stimulation

BMDC or JAWSII DCs (ATCC) were plated at 2×10⁵ per well in a non-tissue culture treated 6-well plate and incubated with 2E′ or PEI at 3 μg/mL or 7 μg/mL. After 24 h incubation, the cells were collected, resuspended in staining buffer, incubated with Fc-blocking antibody for 15 min at 4° C., labeled with anti-mouse CD11c, CD86, CD40, and MHC-II antibodies for 20 min at 4° C., and analyzed by the BD Accuri C6 Flow Cytometer. For measuring cytokine production from the treated BMDC and BMDM, the cells were plated in 96 well plates at a density of 15,000 cells per well and treated with 2E, PEI, 2E′/PTX, 2E′/PTX/siNeg or 2E′/PTX/siPD-L1. After 24 h, the media were analyzed for IL-1β and TNF-α by ELISA (Invitrogen, Carlsbad, CA) according to the manufacturer's protocols.

### TLR-4 and TLR-5 Activation Assays

TLR-4 activation by 2E′ or PEI was evaluated with THP1-XBlue™-MD2-CD14 cells, TLR-4 reporter cells (Invitrogen, Carlsbad, CA). TLR-5 activation was tested with HEK-Blue™ mTLR5 Cells, TLR-5 reporter cells (Invitrogen, Carlsbad, CA). The TLR-4 reporter cells were plated in a 96 well plate at a density of 100,000 cells per well in RPMI medium. The TLR-5 reporter cells were plated in a 96 well plate with a density of 25,000 cells per well in HEK-Blue™ Detection medium. 2E′, PEI, LCA or lipopolysaccharide (LPS) was added to the cells at varying concentrations and incubated for 24 h at 37° C.in 5% CO₂. The supernatant was collected for secreted embryonic alkaline phosphatase (SEAP) assay. The absorbance of SEAP was measured at 620 nm.

### In Vitro Phagocytosis

CT26 or B16F10 cancer cells were stained with Cell Tracker Green (Invitrogen, Carlsbad, CA) for 1 h and treated with 5 μg/mL PTX for 24 h. The PTX-treated cancer cells were collected, counted (2×10⁵), and co-cultured with Cell-Tracker Deep Red (Invitrogen, Carlsbad, CA)-stained BMDC, JAWSII DC or BMDM (2×10⁵) for another 24 h with or without 7 μg/mL of 2E′. The co-cultured cells were collected, resuspended in staining buffer, and analyzed by the BD Accuri C6 Flow Cytometer or BD LSRFortessa Flow Cytometer (San Jose, CA, USA). DCs taking up the PTX-treated cancer cells (cancer cell⁺ DC or macrophages) was quantified as the percentage of double-positive cells in Cell-Tracker Deep Red-stained BMDC, JAWSII DC or BMDM, and the phagocytized cancer cells as the percentage of double-positive cells in Cell Tracker Green-stained cancer cells. For visualizing in vitro cancer cell phagocytosis, the PTX-treated cancer cells (2×10⁵) were co-cultured with Cell-Tracker Deep Red-stained BMDC or BMDM (2×10⁵) for 24 h with or without 7 μg/mL of 2E′ and imaged with the Nikon A1R confocal microscope.

### Cytotoxicity

CT26 cells, B16F10 cells, BMDC, or splenocytes were seeded in a 96 well plate at a density of 8,000 cells per well (CT26, B16F10) or 15,000 cells per well (BMBCs, splenocytes). After 24 h incubation, the cell culture medium was replaced with fresh complete medium containing 2E, PEI, 2E′/PTX or 2E′/PTX/siPD-L1 treatments in different concentrations. After incubation for another 24 h, the cell cytotoxicity was measured by the MTT assay (CT26, B16F10) or propidium iodide (PI) staining (BMDC, splenocytes). For the MTT assay, the treatments were replaced with 100 μL of fresh complete medium and 15 μL of 5 mg/mL MTT solution. After 4 h incubation, 100 μL of stop/solubilization solution was added to the cells and incubated overnight. The absorbance of dissolved formazan was read by the SpectraMax M3 microplate reader (Molecular Devices, Sunnyvale, CA) at 560 nm. For PI staining, the treatments were removed and cells were rinsed with PBS, collected, and resuspended in 100 μL of cell staining buffer. Five microliters (40 ng) of PI staining solution was added to each sample immediately before the analysis by the BD Accuri C6 Flow Cytometer. The combination index (CI) was determined by Compusyn (Combosyn, Inc., Paramus, NJ). The values of CI<1, CI=1, and CI>1 represent synergy, additivity, and antagonism, respectively⁵³.

### Gel Retardation Assay

The formation of 2E′/siPD-L1, 2E′/PTX/siPD-L1 or 2E′/PTX/pDNA complexes was tested by the agarose gel retardation assay. The complexes were prepared varying the weight ratios of 2E′ or 2E′/PTX to siPD-L1 or pDNA. The complexes were loaded in 1.1% agarose gel containing 0.5 μg/mL ethidium bromide in the amounts equivalent to 1 μg siRNA or pDNA and run in 1×TAE buffer at 60 V for 1 h. siRNA or pDNA bands were detected at 302 nm using Azure C300 (Dublin, CA, USA).

### PD-L1 Silencing

CT26 and B16F10 cells were plated in 6-well plates at a density of 10⁵ cells per plate

with 2 mL of culture medium and incubated for 24 h. PD-L1 expression was induced by IFN-γ. To determine the optimal incubation time for PD-L1 expression, the cells were collected at 0, 12, 24, 36 and 48 h after IFN-γ addition, resuspended in staining buffer, incubated with Fc-blocking antibody, stained with anti-mouse PD-L1 antibody, and analyzed by flow cytometry.

To evaluate the silencing effect of siPD-L1 complexes, the cells were incubated in the optimal condition for PD-L1 expression (B16F10 cells with 25 ng/ml of IFN-γ for 4 h and CT 26 cells with 100 ng/ml of IFN-γ for 12 h) and treated with PBS, 2E′/siPD-L1, 2E′/siNeg (siRNA irrelevant to PD-L1 silencing), 2E′/PTX/siPD-L1 or 2E′/PTX/siNeg equivalent to 2.66 μg/mL siRNA in serum-containing medium for 12 h. And then, treatments were replaced with fresh medium and further incubation for 36 h, PD-L1 expression was determined by western blot. The cells were lyzed by cell lysis buffer (Invitrogen, Carlsbad, CA), and the lysates were centrifuged at 12,000 g for 20 min at 4° C. to separate a supernatant. The total protein content in the supernatant was quantified by the BCA assay, and the samples corresponding to 10 mg of protein were mixed with sodium dodecyl sulfate (SDS) gel-loading buffer and heated at 95° C. for 5 min. Samples were separated by 10% SDS-polyacrylamide gel electrophoresis (100 μg proteins per well) and transferred onto polyvinylidene fluoride membrane. The membrane was blocked at room temperature in TBST buffer containing 5% nonfat dried milk (pH 7.4, 20 mM Tris, 150 mM NaCl, and 0.05% Tween 20). After 1 h, the membrane was incubated with anti-mouse PD-L1 and GAPDH antibodies for 24 h at 4° C. per the vendor's recommendation. The membrane was washed three times and incubated with secondary anti-IgG-HRP antibody for 1 h at room temperature. After incubation with the secondary antibody, the membrane was washed three times, and protein bands were detected by Azure C300 (Dublin, CA).

### Cellular Uptake

To examine the cellular uptake of siPD-L1 complexes, 2E′/siPD-L1, Lipofectamine/siPD-L1 and 2E′/PTX/siPD-L1 were prepared with Cy3-labeled siPD-L1. CT26 cells were seeded in Nunc™ glass bottom dishes (Thermo Scientific) at a density of 2×10⁵ and incubated for 24 h. 2E′/siPD-L1, Lipo/siPD-L1 or 2E′/PTX/siPD-L1, equivalent to 66 μg/mL siRNA, in serum-contained medium were incubated with the cells for 4 h or 6 h. The cells were washed, fixed in 4% paraformaldehyde, stained with 200 nM LysoTracker Green and 2 μg/mL Hoechst 33342, and imaged by the Nikon A1R confocal microscope.

### Animal Purchase and Care

All animal procedures were approved by Purdue Animal Care and Use Committee, in conformity with the NIH guidelines for the care and use of laboratory animals. Female Balb/c mice (5-6 week old) and male C57BL/6 (5-6 week old) were purchased from Envigo (Indianapolis, IN, USA) and acclimatized for 1 week prior to the procedure.

### Tumor Retention of 2E′-Cy7 and PEI-Cy7

The retention of intratumorally-injected 2E′-Cy7 and PEI-Cy7 was evaluated in the CT26 model. CT26 tumor cells (5×10⁵) were subcutaneously inoculated in the upper flank of the right hind limb of Balb/c mice. When tumors grew to 100 mm³ on the average, the mice were intratumorally injected with 40 μL of 75 μg/mL 2E′-Cy7 or PEI-Cy7. The fluorescence intensity of 2E′-Cy7 and PEI-Cy7 was monitored by the Spectral Ami Optical Imaging System (Spectral Instruments, Tucson, AZ).

### Antitumor Effects in Balb/c Mice With Bilateral CT26 Tumors

The antitumor effect of 2E′/PTX/siPD-L1 was evaluated in Balb/c mice bearing bilateral CT26 tumors in the flank. Tumors were established in both flanks simultaneously by subcutaneous inoculation. 1×10⁶ of CT26 cells were innoculated in the flank of the right hind limb, and 3×10⁵ of CT26 cells in the left flank of the same mouse. When the tumor on the right side reached 30-50 mm³ on the average, the mice were randomly assigned to different groups to receive 5% dextrose (D5W), 2E′, 2E′/PTX, 2E′/PTX/siNeg or 2E′/PTX/siPD-L1 in the tumor on the right side by intratumoral injection. The sizes of the treated tumor and the non-treated tumor on the left side were measured with a digital caliper every other day, and tumor volumes were calculated as (width²×length)/2.

### Antitumor Effects in Balb/c Mice With CT26 Tumors and Delayed Rechallenge(s) of Live Cells

CT26 tumor cells (5×10⁵) were subcutaneously inoculated in the upper flank of the right hind limb of Balb/c mice. When tumor size reached 30-50 mm³ on the average, the mice were treated with an intratumoral injection of D5W, 2E′, 2E′/PTX, 2E′/PTX/siNeg or 2E′/PTX/siPD-L1. Tumor growth was monitored as described above. To test whether antitumor immunity was established, the mice surviving with complete tumor remission by 30 days from the treatment were rechallenged with 1×10⁵ live CT26 cells on the contralateral flank. The mice resistant to the first rechallenge were challenged again with 2×10⁶ live CT26 cells on 60 days from the treatment. In two separate experiments, surviving Balb/c mice were rechallenged once on 6 or 17 days after the treatment.

2E′/PTX/CDN was tested in Balb/c mice with CT26 tumors. When the tumor grew to 50-100 mm³ on the average, D5W, paclitaxel nanocrystals and free CDN mixture, and 2E′/PTX, or 2E′/PTX/CDN were administered by intratumoral injection, and tumor growth was monitored over 80 days. Tumor-free mice were rechallenged with 1×10⁵ live CT26 cells or 4T1 cells on the contralateral flank on 82 days or 140 days after the treatment.

### Antitumor Effects in B16F10 Tumors of C57BL/6 Mice

B16F10 tumor cells (1×10⁶) were subcutaneously inoculated in the upper flank of the right hind limb of C57BL/6 mice. When tumor size reached ˜150 mm³, the mice were treated with an intratumoral injection of D5W, 2E′, 2E′/PTX, 2E′/PTX/siNeg or 2E′/PTX/siPD-L1. Tumor growth was monitored by measuring the size. Tumor-free mice were rechallenged with 1×10⁵ live B16F10 cells on the contralateral flank.

### Antitumor Effects in C57BL/6 Mice With Bilateral B16F10 Tumors

1×10⁶ and 1×10⁵ B16F10 tumor cells were subcutaneously inoculated in the upper

flank of the right and left hind limb, respectively. When the tumor on the right side grew to 65-100 mm³, it was treated with single intratumoral injection of D5W (n=6), 2E′/siPD-L1+PTX NC (n=8), 2E′/PTX/siNeg (n=9) and 2E′/PTX/siPD-L1 (n=9). The growth of treated and untreated tumors was monitored by measuring the size.

### Antitumor Effects in 4T1 Orthotopic Tumors of Balb/c Mice

4T1-Luc cell line was a gift from Prof. Michael Wendt at Purdue University. 4T1-Luc 2.5×10⁴ were inoculated in the mammary fat pad of female Balb/c mice. When tumor size reached ˜50 mm³, D5W, 2E′/PTX/siNeg, or 2E′/PTX/siPD-L1 were administered by intratumoral injection, or the tumor was removed by partial or complete surgical resection. Tumor growth was monitored by measuring the size. In addition, whole body imaging was performed by the Spectral Ami Optical Imaging System (Spectral Instruments, Tucson, AZ) to determine the luciferase expression in lieu of tumor growth. Tumor-free mice were rechallenged with 2.5×10³ live 4T1-Luc cells on the contralateral mammary gland.

### Isolation of Splenocytes

The spleen was collected from healthy or tumor-bearing mice to isolate splenocytes. The collected spleens were cut into pieces and filtered through 70 μm and 40 μm cell strainers sequentially to obtain a single-cell suspension. The cell suspension was incubated with 1 mL ammonium-chloride-potassium (ACK) lysis buffer for 3 min to remove red blood cells.

### Tumor-Specific Immunity

The single cell suspension of splenocytes was stained with zombie dye, incubated with anti-mouse CD16/32 antibody to block non-specific binding of the immunoglobulin to Fc receptors, and then labeled with fluorochrome-conjugated antibodies: iTAg Tetramer/APC-H-2 Kb TRP2 (SVYDFFVWL, SEQ ID NO:5), FITC anti-mouse CD8 antibody (KT15), and PE anti-mouse CD3 antibody (17A2), The labeled cells were analyzed by BD LSRFortessa Flow Cytometer.

Splenocytes collected from B16F10 tumor-bearing mice were challenged with MHC-I-restricted peptide antigen Trp2₁₈₀₋₁₈₈ (SVYDFFVWL, SEQ ID NO:5) to determine the response. Splenocytes were suspended in MEM-alpha medium supplemented with 100 units/mL penicillin, 100 μg/mL streptomycin, 10 mM β-mercaptoethanol, 20 ng/ml murine GM-CSF, and 20% fetal bovine serum were seeded at 1×10⁶ cells per well in a 96 well plate and stimulated with 1-5 μg/mL of Trp2 peptides. After 48 h incubation, the cells were centrifuged at 500 ref for 8 min to collect the supernatant. The IFN-γ concentration in each supernatant was measured by ELISA (Biolegend, San Diego, CA, USA) and compared with that of the non-challenged cells collected from the same mouse.

### Immunophenotyping of Tumors and Lymphoid Organs

Tumors were collected from C57BL/6 mice with B16F10 tumors on 7 days after the treatment, treated with 2 mg/mL collagenase type IV. 0.2 mg/mL DNase I, and 0.2 mg/mL hyaluronidase for 2 h at 37° C., and ground with the rubber end of a syringe plunger. The cell suspension was filtered through 70 μm and 40 μm cell strainers sequentially and centrifuged at 500×g for 8 min. Red blood cells were removed with ACK lysis buffer. The single cell suspension was stained with zombie dye and incubated with anti-mouse CD16/32 antibody to block non-specific binding of the immunoglobulin to Fc receptors and then labeled with fluorochrome-conjugated antibodies: FITC anti-mouse CD3 antibody (17A2), PE anti-mouse CD4 antibody (RM4-5), APC anti-mouse CD8a antibody (53-6.7), FITC anti-mouse CD11c antibody (N418), APC anti-mouse CD86 antibody (GL-1), APC anti-mouse CD40 antibody (3/23), APC anti-mouse MHC-II antibody (M5/114.15.2), or FITC anti-mouse F4/80 antibody (BM8). The labeled cells were analyzed by the BD Accuri C6 Flow Cytometer or BD LSRFortessa Flow Cytometer.

### RNA Sequencing Assay

Generation of libraries and sequencing: When B16F10 tumors in C57BL/6 mice grew to ˜150 mm³, the mice received an intratumoral injection of D5W, 2E′/PTX/siNeg or 2E′/PTX/siPD-L1 (day 0). Tumors were collected from the untreated animals on day 0 or from the treated animals on day 7 and digested to a single cell suspension. Cell pellets were lysed in 2 mL of TRK lysis buffer and stored frozen until all samples had been collected. Each TRK lysate 700 μL was moved forward to RNA isolation using the RNeasy extraction kit (Qiagen). RNA concentration was determined by Nanodrop. One microgram of RNA was used to generate libraries with the Universal Plus mRNA-Seq kit (Tecan) per manufacturer instructions. A single Illumina NovaSeq 6000 S4 300 cycle, v1.5 chemistry, lane was clustered with a pool of the libraries to produce paired-end 2×150 base reads.

Adapter and Quality Trimming of Reads: The program fastp v.0.12.5 was used to further trim reads based on quality score and to remove adapter sequences⁵⁴. The minimum quality score was set to 30, and reads shorter than 50 bases or that were unpaired after trimming were discarded. STAR v. 2.5.4b was used to align reads to the Ensembl Mus musculus genome database version GRCm38.p6 using-twopassMode Basic, modifying the tag HI in the BAM alignment file to start at 0, and removing noncanonical splice junctions⁵⁵. The Subread v.2.0.2 software module featureCounts on stranded mode was used to tabulate reads mapping to genes into a gene count matrix using Ensembl Mus musculus genome annotations⁵⁶.

Statistical analyses: The Bioconductor package edgeR v.3.24.3 was used to fit a quasi-likelihood negative binomial generalized log-linear model to the count data, followed by genewise statistical tests to identify differentially expressed genes^{57,58}. The Benjamini-Hochberg false discovery rate correction is used to correct p-values for multiple testing⁵⁹. The Bioconductor packages BiomaRt v. 2.38.0⁶⁰ and ClusterProfiler v 3.10.1⁶¹ were used in the annotation of genes and in performing pathway and gene ontology enrichment analyses on the differentially expressed genes (results were deemed significant if the adjusted p-value<0.05).

Histology

Organs (heart, liver, spleen, lung, kidney and tumors) from C57BL/6 mice with B16F10 tumors were collected on 7 days after the treatment, fixed in 10% neutral buffered formalin and sectioned at a thickness of 4 μm. Heart, liver, spleen, lung and kidney sections were stained with H&E, and tumor sections were stained with rat anti-mouse CD8a monoclonal antibody (eBioscience, clone 4SM15) followed by goat anti-rat secondary antibody (Vector Labs, MP-5444) or with rabbit anti-mouse PD-L1 antibody (Novus biologicals, clone 2096A) followed by horse anti-rabbit secondary antibody (Vector Labs, MP-5401). Slides were rinsed twice in tris buffered saline with Tween® 20, applied with vector immPACT red (Vector Labs, SK-5105) for 20 min and transferred to a Leica Autostainer XL for hematoxylin counterstain, dehydration and coverslipping. Images were taken using a Leica Versa8 whole-slide scanner.

Statistical Analysis

All statistical analyses were performed with GraphPad Prism 9 (La Jolla, CA). All data were analyzed with one-way or two-way ANOVA test to determine the statistical difference of means among various groups, followed by the recommended multiple comparisons tests. A value of p<0.05 was considered statistically significant.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected. It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES

1. Hodi, F. S. et al. Improved Survival with Ipilimumab in Patients with Metastatic Melanoma. New England Journal of Medicine 363, 711-723 (2010).
2. Grupp, S. A. et al. Chimeric Antigen Receptor-Modified T Cells for Acute Lymphoid Leukemia. New England Journal of Medicine 368, 1509-1518 (2013).
3. Upadhaya, S., Hubbard-Lucey, V. M. & Yu, J. X. Immuno-oncology drug development forges on despite COVID-19. Nat Rev Drug Discov 19, 751-752 (2020).
4. Tan, S., Li, D. & Zhu, X. Cancer immunotherapy: Pros, cons and beyond. Biomedicine & Pharmacotherapy 124, 109821 (2020).
5. Kim, G. B. et al. Xenogenization of tumor cells by fusogenic exosomes in tumor microenvironment ignites and propagates antitumor immunity. Science Advances 6, eaaz2083 (2020).
6. Aznar, M. A. et al. Intratumoral Delivery of Immunotherapy—Act Locally, Think Globally. The Journal of Immunology 198, 31 (2017).
7. Martins, F. et al. Adverse effects of immune-checkpoint inhibitors: epidemiology, management and surveillance. Nature Reviews Clinical Oncology 16, 563-580 (2019).
8. Champiat, S. et al. Intratumoral Immunotherapy: From Trial Design to Clinical Practice. Clin Cancer Res 27, 665-679 (2021).
9. Lau, T. S. et al. Paclitaxel Induces Immunogenic Cell Death in Ovarian Cancer via TLR4/IKK2/SNARE-Dependent Exocytosis. Cancer Immunology Research 8, 1099-1111 (2020).
10. Sun, F. et al. Oxaliplatin induces immunogenic cells death and enhances therapeutic efficacy of checkpoint inhibitor in a model of murine lung carcinoma. J Recept Signal Transduct Res 39, 208-214 (2019).
11. Chuang, Y .-C. et al. Adjuvant Effect of Toll-Like Receptor 9 Activation on Cancer Immunotherapy Using Checkpoint Blockade. Front Immunol 11 (2020).
12. Kwak, G. et al. Programmed Cell Death Protein Ligand-1 Silencing with Polyethylenimine-Dermatan Sulfate Complex for Dual Inhibition of Melanoma Growth. ACS Nano 11, 10135-10146 (2017).
13. Tahamtan, A., Salimi, V. et al. Anti-Inflammatory MicroRNAs and Their Potential for Inflammatory Diseases Treatment. Front Immunol 9 (2018).
14. Chen, Q. et al. In situ sprayed bioresponsive immunotherapeutic gel for post-surgical cancer treatment. Nat Nanotechnol 14, 89-97 (2019).
15. Park, J. et al. Combination delivery of TGF-β inhibitor and IL-2 by nanoscale liposomal polymeric gels enhances tumour immunotherapy. Nat Mater 11, 895-905 (2012).
16. Miao, L. et al. Delivery of mRNA vaccines with heterocyclic lipids increases anti-tumor efficacy by STING-mediated immune cell activation. Nature Biotechnology 37, 1174-1185 (2019).
17. Li, A. W. et al. A facile approach to enhance antigen response for personalized cancer vaccination. Nature Materials 17, 528-534 (2018).
18. Li, Z. et al. Targeting pulmonary tumor microenvironment with CXCR4-inhibiting nanocomplex to enhance anti-PD-L1 immunotherapy. Science Advances 6, eaaz9240 (2020).
19. Wang, F. et al. Tumour sensitization via the extended intratumoural release of a STING agonist and camptothecin from a self-assembled hydrogel. Nature Biomedical Engineering (2020).
20. Pandey, A. P. & Sawant, K. K. Polyethylenimine: A versatile, multifunctional non-viral vector for nucleic acid delivery. Materials Science and Engineering: C 68, 904-918 (2016).
21. Cubillos-Ruiz, J. R. et al. Polyethylenimine-based siRNA nanocomplexes reprogram tumor-associated dendritic cells via TLR5 to elicit therapeutic antitumor immunity. The Journal of Clinical Investigation 119, 2231-2244 (2009).
22. Holtmann, T. M. et al. Bile Acids Activate NLRP3 Inflammasome, Promoting Murine Liver Inflammation or Fibrosis in a Cell Type-Specific Manner. Cells 10 (2021).
23. Wang, J., Meng, F., & Yeo, Y. In-vitro and in-vivo difference in gene delivery by lithocholic acid-polyethyleneimine conjugate. Biomaterials 217, 119296 (2019).
24. Le, W., Chen, B., Cui, Z., Liu, Z. & Shi, D. Detection of cancer cells based on glycolytic-regulated surface electrical charges. Biophysics Reports 5, 10-18 (2019).
25. Chen, B. et al. Targeting Negative Surface Charges of Cancer Cells by Multifunctional Nanoprobes. Theranostics 6, 1887-1898 (2016).
26. Chollet, P., Favrot, M. C., Hurbin, A. & Coll, J. L. Side-effects of a systemic injection of linear polyethylenimine-DNA complexes. J Gene Med 4, 84-91 (2002).
27. Wegmann, F. et al. Polyethyleneimine is a potent mucosal adjuvant for viral glycoprotein antigens. Nature Biotechnology 30, 883 (2012).
28. Eisenbarth, S. C., Colegio, O. R., O'Connor, W., Sutterwala, F. S. & Flavell, R. A. Crucial role for the Nalp3 inflammasome in the immunostimulatory properties of aluminium adjuvants. Nature 453, 1122-1126 (2008).
29. Gardai, S. J. et al. Cell-surface calreticulin initiates clearance of viable or apoptotic cells through trans-activation of LRP on the phagocyte. Cell 123, 321-334 (2005).
30. Garg, A. D., Dudek-Peric, A. M., Romano, E. & Agostinis, P. Immunogenic cell death. Int J Dev Biol 59, 131-140 (2015).
31. Kroemer, G., Galluzzi, L., Kepp, O. & Zitvogel, L. Immunogenic cell death in cancer therapy. Annu Rev Immunol 31, 51-72 (2013).
32. Janicka, M. & Gubernator, J. Use of nanotechnology for improved pharmacokinetics and activity of immunogenic cell death inducers used in cancer chemotherapy. Expert Opinion on Drug Delivery 14, 1059-1075 (2017).
33. Chiarella, P., Bruzzo, J., Meiss, R. P. & Ruggiero, R. A. Concomitant tumor resistance. Cancer Letters 324, 133-141 (2012).
34. Joo, H. G. Altered maturation of dendritic cells by taxol, an anticancer drug. J Vet Sci 4, 229-234 (2003).
35. Chen, H. et al. The promotion of type 1 T helper cell responses to cationic polymers in vivo via toll-like receptor-4 mediated IL-12 secretion. Biomaterials 31, 8172-8180 (2010).
36. Fromen, C. A. et al. Controlled analysis of nanoparticle charge on mucosal and systemic antibody responses following pulmonary immunization. Proc Natl Acad Sci USA 112, 488-493 (2015).
37. Wei, X. et al. Cationic nanocarriers induce cell necrosis through impairment of Na(+)/K(+)-ATPase and cause subsequent inflammatory response. Cell Res 25, 237-253 (2015).
38. Openshaw, P. J. M. & Tregoning, J. S. Immune responses and disease enhancement during respiratory syncytial virus infection. Clin Microbiol Rev 18, 541-555 (2005).
39. Fleming, V. et al. Melanoma Extracellular Vesicles Generate Immunosuppressive Myeloid Cells by Upregulating PD-L1 via TLR4 Signaling. Cancer Res 79, 4715-4728 (2019).
40. Cai, J., Wang, D., Zhang, G. & Guo, X. The Role Of PD-1/PD-L1 Axis In Treg Development And Function: Implications For Cancer Immunotherapy. Onco Targets Ther 12, 8437-8445 (2019).
41. Parkhurst, M. R. et al. Identification of a Shared <em>HLA-A*0201</em>-restricted T-Cell Epitope from the Melanoma Antigen Tyrosinase-related Protein 2 (TRP2). Cancer Research 58, 4895-4901 (1998).
42. Park, J., Sun, B. & Yeo, Y. Albumin-coated nanocrystals for carrier-free delivery of paclitaxel. J Control Release 263, 90-101 (2017).
43. Park, J. et al. A Comparative In Vivo Study of Albumin-Coated Paclitaxel Nanocrystals and Abraxane. Small 14, e1703670 (2018).
44. Ravindranathan, S. et al. Tumor-derived granulocyte colony-stimulating factor diminishes efficacy of breast tumor cell vaccines. Breast Cancer Research 20, 126 (2018).
45. Schrors, B. et al. Multi-Omics Characterization of the 4T1 Murine Mammary Gland Tumor Model. Frontiers in Oncology 10 (2020).
46. Pulaski, B. A. & Ostrand-Rosenberg, S. Mouse 4T1 Breast Tumor Model. Current Protocols in Immunology 39, 20.22.21-20.22.16 (2000).
47. Foote, J. B. et al. A STING Agonist Given with OX40 Receptor and PD-L1 Modulators Primes Immunity and Reduces Tumor Growth in Tolerized Mice. Cancer Immunol Res 5, 468-479 (2017).
48. Barber, G. N. STING: infection, inflammation and cancer. Nat Rev Immunol 15, 760-770 (2015).
49. Lu, X. et al. Engineered PLGA microparticles for long-term, pulsatile release of STING agonist for cancer immunotherapy. Science Translational Medicine 12, eaaz6606 (2020).
50. Gadkaree, S. K. et al. Induction of tumor regression by intratumoral STING agonists combined with anti-programmed death-L1 blocking antibody in a preclinical squamous cell carcinoma model. Head & Neck 39, 1086-1094 (2017).
51. Cherukula, K., Bae, W. K., Lee, J. H. & Park, I. K. Programmed ‘triple-mode’ anti-tumor therapy: Improving peritoneal retention, tumor penetration and activatable drug release properties. Biomaterials 169, 45-60 (2018).
52. Feng, M. et al. Stabilization of a hyaluronate-associated gene delivery system using calcium ions. Biomaterials Science 2, 936-942 (2014).
53. Chou, T. C. Theoretical basis, experimental design, and computerized simulation of synergism and antagonism in drug combination studies. Pharmacol Rev 58, 621-681 (2006).
54. Chen, S., Zhou, Y., Chen, Y. & Gu, J. fastp: an ultra-fast all-in-one FASTQ preprocessor. Bioinformatics 34, 1884-1890 (2018).
55. Dobin, A. et al. STAR: ultrafast universal RNA-seq aligner. Bioinformatics 29, 15-21 (2012).
56. Liao, Y., Smyth, G. K. & Shi, W. featureCounts: an efficient general purpose program for assigning sequence reads to genomic features. Bioinformatics 30, 923-930 (2013).
57. Robinson, M. D., McCarthy, D. J. & Smyth, G. K. edger: a Bioconductor package for differential expression analysis of digital gene expression data. Bioinformatics 26, 139-140 (2009).
58. McCarthy, D. J., Chen, Y. & Smyth, G. K. Differential expression analysis of multifactor RNA-Seq experiments with respect to biological variation. Nucleic Acids Research 40, 4288-4297 (2012).
59. Benjamini, Y. & Hochberg, Y. Controlling the False Discovery Rate: A Practical and Powerful Approach to Multiple Testing. Journal of the Royal Statistical Society. Series B (Methodological) 57, 289-300 (1995).
60. Durinck, S. et al. BioMart and Bioconductor: a powerful link between biological databases and microarray data analysis. Bioinformatics 21, 3439-3440 (2005).
61. Yu, G., Wang, L.-G., Han, Y. & He, Q.-Y. clusterProfiler: an R Package for Comparing Biological Themes Among Gene Clusters. OMICS: A Journal of Integrative Biology 16, 284-287 (2012).

What is claimed is:

1. A composition matter as an antitumor immunotherapy or a diagnosis tool comprising a polyethyleneimine derivative as an immunoadjuvant and a chemotherapeutic drug or a hydrophobic molecule.

2. The composition matter according to claim 1 further comprising a microRNA, messenger RNA, plasmid DNA, small interfering RNA (siRNA), oligonucleotide, or a cyclic dinucleotide.

3. The composition matter according to claim 2, wherein said siRNA is PD-L1 siRNA.

4. The composition matter according to claim 1, wherein said chemotherapeutic drug or a hydrophobic molecule is paclitaxel, sorafenib, itraconazole, docetaxel, doxorubicin, bortezomib, carfilzomib, camptothecin, cisplatin, oxaliplatin, cytarabine, vincristine, irinotecan, amphotericin, niflumic acid, probucol, indomethacin, gemcitabine, or a pharmaceutically acceptable salt thereof, or a hydrophobic dye or a salt thereof.

5. The composition matter according to claim 4, wherein said hydrophobic dye is a hydrophobic fluorescent dye comprising DiR′; DiIC18(7) (1,1′-Dioctadecyl-3,3,3′,3′ Tetramethylindotricarbocyanine Iodide), Cyanine7, Cyanine 5, or an acceptable salt thereof.

6. The composition matter according to claim 1, wherein said polyethyleneimine derivative is a modified/conjugated polyethyleneimine by lithocholic acid (LCA), cholic acid, glycocholic acid, taurocholic acid, deoxycholic acid, chenodeoxycholic acid, glycochenodeoxycholic acid, taurochenodeoxycholic acid, or an acceptable salt thereof.

7. The composition matter according to claim 1, wherein said polyethyleneimine has a molecular weight range of about 2,500 Da to about 250,000 Da.

8. The composition matter according to claim 1 as an antitumor immunotherapy, wherein said composition matter is administered intratumorally.

9. The composition matter according to claim 1 as an antitumor immunotherapy, wherein said composition matter is administered systemically.

10. A pharmaceutical composition comprising the composition matter according to claim 1, together with one or more diluents, excipients, or carriers.

11. A method for treating a subject with cancer comprising the step of administrating a therapeutic effective amount of a composition comprising a polyethyleneimine derivative as an immunoadjuvant and an antitumor agent to the subject in need of relief from said cancer.

12. The method according to claim 11 further comprising a microRNA, messenger RNA, plasmid DNA, small interfering RNA (siRNA), oligonucleotide, or a cyclic dinucleotide.

13. The method according to claim 12, wherein said siRNA is PD-L1 siRNA.

14. The method according to claim 11, wherein said chemotherapeutic drug is a hydrophobic chemotherapeutic molecule.

15. The method according to claim 14, wherein said hydrophobic chemotherapeutic drug comprises paclitaxel, sorafenib, itraconazole, docetaxel, doxorubicin, bortezomib, carfilzomib, camptothecin, cisplatin, oxaliplatin, cytarabine, vincristine, irinotecan, amphotericin, niflumic acid, probucol, indomethacin, gemcitabine, or a pharmaceutically acceptable salt thereof.

16. The method according to claim 11, wherein said polyethyleneimine derivative is a wherein said polyethyleneimine derivative is a modified/conjugated polyethyleneimine by lithocholic acid (LCA), cholic acid, glycocholic acid, taurocholic acid, deoxycholic acid, chenodeoxycholic acid, glycochenodeoxycholic acid, taurochenodeoxycholic acid, or an acceptable salt thereof.

17. The method according to claim 11, wherein said polyethyleneimine has a molecular weight range of about 2,500 Da to about 250,000 Da.

18. The method according to claim 11 as an antitumor immunotherapy, wherein said composition matter is administered intratumorally.

19. The method according to claim 11 as an antitumor immunotherapy, wherein said composition matter is administered systemically.

20. The composition matter according to claim 1, wherein said composition exists in a filament form.

21. A composition matter for diagnosis purpose comprising a polyethyleneimine derivative and a hydrophobic dye.


SEQUENCE LISTING (5 sequences)

SEQ ID NO: 1 - DNA, 21 bp, Artificial Sequence
  siRNA specific for pdcd1lg1 mRNA, sense
cccacauaaaaaacaguugtt

SEQ ID NO: 2 - DNA, 21 bp, Artificial Sequence
  siRNA specific for mouse pdcd1lg1 mRNA, antisense
caacuguuuuuuaugugggtt

SEQ ID NO: 3 - DNA, 23 bp, Artificial sequence
  negative siRNA, sense
ugaaguugcacuugaagucdtdt

SEQ ID NO: 4 - DNA, 23 bp, Artificial sequence
  negative siRNA, antisense
gacuucaagugcaacuucadtdt

SEQ ID NO: 5 - protein, 9 aa, Artificial sequence
  peptide antigen Trp2 (180-188)
SVYDFFVWL